(19) United States
(12) Patent Application Publication
(10) Pub. No.: US 2016/0208323 A1
(43) Pub. Date: Jul. 21, 2016

(54) Methods for Shearing and Tagging DNA for Chromatin Immunoprecipitation and Sequencing

(71) Applicants: THE BROAD INSTITUTE, INC., Cambridge, MA (US); PRESIDENT AND FELLOWS OF HARVARD COLLEGE, Cambridge, MA (US); THE GENERAL HOSPITAL CORP (DBA MASSACHUSETTS GENERAL HOSPITAL), Boston, MA (US); MASSACHUSETTS INSTITUTE OF TECHNOLOGY, Cambridge, MA (US)

(72) Inventors: Bradley BERNSTEIN, Cambridge, MA (US); Alon GOREN, Cambridge, MA (US); Chad NUSBAUM, Newton, MA (US); Oren RAM, Chestnut Hill, MA (US); Assaf ROTEM, Cambridge, MA (US); Daniel TARJAN, Cambridge, MA (US); Jeffrey XING, Clarksville, MD (US); Aviv REGEV, Cambridge, MA (US)

(21) Appl. No.: 14/900,217

(22) PCT Filed: Jun. 19, 2014
(86) PCT No.: PCT/US2014/043295

§ 371 Date: Dec. 21, 2015

Related U.S. Application Data
(60) Provisional application No. 61/838,036, filed on Jun. 21, 2013.

(51) Int. Cl. C12Q 1/68; G01N 33/541; C12N 15/10

(52) U.S. Cl. CPC C12Q 1/6874; C12N 15/1082; G01N 33/541

(57) ABSTRACT

Disclosed are methods for shearing and tagging chromatin DNA. The disclosed methods include contacting chromatin DNA with at least one transposome, that includes a transposase enzyme. The transposon is made up of a first DNA molecule that includes a first transposase recognition site and a second DNA molecule that includes a second transposase recognition site, wherein the transposase integrates the first and second DNA molecules into chromatin DNA. The first and second DNA molecules of the transposon can be disconnected, such that upon integration of the transposon the chromatin bound DNA is sheared and tagged with the first and second DNA molecules, for example to prepare a library of sheared and tagged chromatin DNA fragments.

### CROSS-REFERENCE TO RELATED APPLICATION

This application claims the priority benefit of the earlier filing date of U.S. Provisional Application No. 61/838,036, filed Jun. 21, 2013, which is hereby incorporated herein in its entirety.

### STATEMENT OF GOVERNMENT SUPPORT

This invention was made with government support under grant number U54HG004570 awarded by the National Human Genome Research Institute and grant number U54HG006991 awarded by the National institutes of Health. The government has certain rights in the invention.

### FIELD OF THE DISCLOSURE

This disclosure relates to the field of biochemistry and specifically to methods of shearing and tagging immunoprecipitated chromatin DNA and the sequencing of sheared and tagged DNA.

### BACKGROUND

Chromatin immuno-precipitation (ChIP) is a powerful tool for evaluating interaction of proteins with specific genomic DNA regions in vivo, to provide a better understanding of the mechanisms of gene regulation, DNA replication, and DNA repair. Typically ChIP involves fixative treatment of live cells with a chemical cross-linker to cross-link any DNA-bound proteins. The cells are then lysed, and the chromatin released from the cells is sheared mechanically or enzymatically, in order to reduce fragment size and increase resolution. The resultant sheared complexes are then immuno-precipitated with antibodies specific to the protein of interest, and the DNA fragments are analyzed, e.g., using real time PCR, sequencing, or microarray hybridization.

Specific DNA sites in direct physical interaction with transcription factors and other proteins can be isolated by chromatin immuneprecipitation to produce a library of target DNA sites bound to a protein of interest in vivo. With the advent of massively parallel sequence, the libraries can be rapidly analyzed, and mapped to whole-genome sequence databases to determine the interaction pattern of any protein with DNA, or the pattern of any epigenetic chromatin modifications. This can be applied to the set of ChIP-able proteins and modifications, such as transcription factors, polymerases and transcriptional machinery, structural proteins, protein modifications, and DNA modifications. ChIP sequencing (ChIP-seq) can be used to determining how proteins interact with DNA, for example to regulate gene expression. ChIP-seq technology is currently seen primarily as an alternative to ChIP-chip which requires a hybridization array. This necessarily introduces some bias, as an array is restricted to a fixed number of probes.

Because of the vast amount of information that can be obtained from ChIP it can limited by the ability to sequence immunoprecipitated DNA, for example in limited numbers of primary cell, improved methods or ChIP-Seq are needed. This disclosure meets those needs.

### SUMMARY OF THE DISCLOSURE

Disclosed are methods for shearing and tagging chromatin DNA. The disclosed methods include contacting chromatin DNA with at least one transposome, that includes a transposase enzyme, such as a Tn5 transposase, Mu transposase IS5 or an IS91 transposase and a transposon. The transposon is made up of a first DNA molecule that includes a first transposase recognition site and a second DNA molecule that includes a second transposase recognition site, wherein the transposase integrates the first and second DNA molecules into chromatin DNA. The first and second DNA molecules of the transposon can by disconnected, such that upon integration of the transposon the chromatin bound DNA is shearing and tagged with the first and second DNA molecules, for example to preparing a library of sheared and tagged chromatin DNA fragments. The chromatin for use in the disclosed methods can be provided as cross-linked chromatin, for example by cross-linking chromatin to cross-link chromatin associated factors to chromatin DNA.

In some embodiments, a chromatin-associated factor cross-linked to the nucleic acid with a specific binding agent that specifically binds to the chromatin-associated factor, for example to immunoprecipitate the chromatin.

In some embodiments of the method, the first and/or second DNA molecule further include a barcode. In some embodiments of the method the first and/or second DNA molecule include a sequencing adaptor. While in still other embodiments of the method, the first and/or second DNA molecule include a universal priming site.

In some embodiments, the chromatin DNA is contacted with at least two different transposomes, and wherein the different transposomes integrate different DNA sequences into the chromatin DNA.

Also disclosed are kits that can be used for the disclosed methods.

### BRIEF DESCRIPTION OF THE FIGURES

FIG. 1 is a flow diagram showing an embodiment of the methods disclosed herein.

FIG. 2 is a digital image of a nucleic acid gel. PCR amplification of mouse embryonic stem-cell (mES) chromatin from cell lysate was tagmented in different volumes with 10 ul carried over into Nextera® reaction. This varied the concentration of detergent in the Nextera® reaction. The last lane is a positive control where mES gDNA isolated with a DNA extraction kit was tagmented with the Nextera® kit per manufacturer protocol. Recognizing the importance of detergent as an inhibitor of transposase activity, it was determined that decreasing detergent concentration in the Nextera® tagmentation reaction improved tagmentation as determined by amplifying tagmented genomic DNA.

FIG. 3 is a digital image of a nucleic acid gel. To account for the possibility that the 55° C. temperature of the Nextera® reaction may have been dissociating the DNA molecules from histones the technique was repeated at lower temperatures. PCR amplification of chromatin and naked DNA tagmented at 37° C. for 1 hour instead of 55° C. for 5 min. Results are comparable to 55° C. reaction. Compare lane 2 to FIG. 2, lane 4.

FIG. 4 is a digital image of a nucleic acid gel. PCR amplification of DNA isolated from chromatin that was tagmented at 37° C. for 1 hour and then immunoprecipitated using an antibody for histone 3, lysite 4 trimethylation. Samples from both mES and K562 chromatin show good amplification if tagmented, and not if tagmentation reaction is performed in the absence of transposase. Laddering is visible in lanes 2 and 3, implying that the transposase acted mainly on internucleosomal regions.

FIG. 5 is a digital image of a nucleic acid gel. To test the ability for the technique to operate on heterochromatinized regions of the genome the experiments were repeated where immunoprecipitation targeted histone 3, lysine 36 trimethylation and histone 3, lysine 27 trimethylation. PCR amplification as in FIG. 4 of tagmented mES chromatin immunoprecipitated using antibodies to H3K36me3, H3K27me3 in addition to H3K4me3.

FIGS. 6A and 6B are plots. Computational analysis of sequencing of the DNA isolated by this method shows promising agreement with data obtained by the current standard protocol for ChIP-seq. Comparing genomic bins aggregating sequencing data for mES H3K4me3 regions genome-wide for bulk ChIP-seq using MNase followed by adapter ligation versus Nextera-ChIP-seq demonstrates good agreement between the two protocols. Using 5 kilobase or 3 kilobase bins yields an R̂2 of 0.61 or 0.59, respectively. (FIG. 6A) Comparing only known promoter regions, where H3K4me3 is known to be prevalent, demonstrates an even better agreement. Using 5 kilobase binning of the sequencing data yields at R̂2 of 0.66. (FIG. 6B)

FIG. 7 is a trace showing the results of a ing H3K4me3 ChIP->Nextera® library prep.

FIG. 8 is a trace showing the results of 0.01 ng H3K4me3 ChIP->Nextera® library prep.

FIG. 9 is a comparison of the disclosed methods on using NexteraXT® on low input ChIP.

FIG. 10 is a heatmap showing a comparison of varying Nextera® libraries with existing sequencing data—H3K9me3, 10 kb bins, genome-wide.

FIG. 11 is a scatter plot showing K9me3 10 kb bins avg signal ENCODE bio-rep.

FIG. 12 is a scatter plot showing K9me3 10 KB bins avg signal Nextera® vs NEB library; technical-rep.

FIG. 13 is a scatter plot showing K9me3 10 KB bins avg signal Nextera® 1 ng vs Nextera® 0.01 ng; technical-rep.

FIG. 14 is a scatter plot showing K9me3 10 KB bins avg signal Nextera® 1 ng vs NEB library; bio-rep.

FIG. 15 is a heatmap showing a comparison of varying Nextera libraries with existing sequencing data—H3K4me3, 5 kb bins, genome-wide.

FIG. 16 is a scatter plot showing K4me3 5 KB bins avg signal ENCODE bio-rep.

FIG. 17 is a scatter plot showing K4me3 5 KB bins avg signal Nextera® vs NEB library; technical-rep.

FIG. 18 is K4me3 5 KB bins avg signal Nextera® vs NEB library; technical-rep.

FIG. 19 is a flow diagram showing an embodiment of the methods disclosed herein.

### DETAILED DESCRIPTION OF SEVERAL EMBODIMENTS

### I. Summary of Terms

Unless otherwise noted, technical terms are used according to conventional usage. Definitions of common terms in molecular biology may be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710). The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will control.

To facilitate review of the various embodiments of this disclosure, the following explanations of specific terms are provided.

Antibody: A polypeptide ligand that includes at least a light chain or heavy chain immunoglobulin variable region and specifically binds an epitope of an antigen, such as an epitope on a protein associated with chromatin DNA. Antibodies can include monoclonal antibodies, polyclonal antibodies, or fragments of antibodies.

The term “specifically binds” refers to, with respect to an antigen, the preferential association of an antibody or other ligand, in whole or part, with a specific polypeptide, such as a specific protein bound to chromatin DNA, for example a transcription factor. A specific binding agent binds substantially only to a defined target. It is recognized that a minor degree of non-specific interaction may occur between a molecule, such as a specific binding agent, and a non-target polypeptide. Nevertheless, specific binding can be distinguished as mediated through specific recognition of the antigen. Although selectively reactive antibodies bind antigen, they can do so with low affinity. Specific binding typically results in greater than 2-fold, such as greater than 5-fold, greater than 10-fold, or greater than 100-fold increase in amount of bound antibody or other ligand (per unit time) to a target polypeptide, such as compared to a non-target polypeptide. A variety of immunoassay formats are appropriate for selecting antibodies specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

Antibodies can be composed of a heavy and a light chain, each of which has a variable region, termed the variable heavy (VH) region and the variable light (VL) region. Together, the VH region and the VL region are responsible for binding the antigen recognized by the antibody. This includes intact immunoglobulins and the variants and portions of them well known in the art, such as Fab′ fragments, F(ab)′2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes recombinant forms such as chimeric antibodies (for example, humanized murine antibodies), heteroconjugate antibodies (such as bispecific antibodies). See also, Pierce Catalog and Handbook, 1994-1995 (Pierce Chemical Co., Rockford, Ill.); Kuby, Immunology, 3rd Ed., W.H. Freeman & Co., New York, 1997.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. These fused cells and their progeny are termed “hybridomas.” Monoclonal antibodies include humanized monoclonal antibodies.

Amplification: To increase the number of copies of a nucleic acid molecule, such as ChIP nucleic acids. The resulting amplification products are called “amplicons.” Amplification of a nucleic acid molecule (such as a DNA or RNA molecule) refers to use of a technique that increases the number of copies of a nucleic acid molecule (including fragments).

An example of amplification is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

Other examples of in vitro amplification techniques include quantitative real-time PCR; reverse transcriptase PCR (RT-PCR); real-time PCR (rt PCR); real-time reverse transcriptase PCR (rt RT-PCR); nested PCR; strand displacement amplification (see U.S. Pat. No. 5,744,311); transcription-free isothermal amplification (see U.S. Pat. No. 6,033,881, repair chain reaction amplification (see WO 90/01069); ligase chain reaction amplification (see European patent publication EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134) amongst others.

Binding or stable binding: An association between two substances or molecules, such as the hybridization of one nucleic acid molecule to another or itself, the association of an antibody with a peptide, or the association of a protein with another protein (for example the binding of a transcription factor to a cofactor) or nucleic acid molecule (for example the binding of a transcription factor to a nucleic acid, such as chromatin DNA).

Binding site: A region on a protein, DNA, or RNA to which other molecules stably bind. In one example, a binding site is the site on a DNA molecule, such as chromatin DNA, that a chromatin associated factor, such as a transcription factor, binds (referred to as a transcription factor binding site).

Contacting: Placement in direct physical association, for example both in solid form and/or in liquid form. Contacting can occur in vitro with isolated cells or cell lysates, or in vivo by administering to a subject.

Control: A reference standard. A control can be a known value or range of values indicative of basal levels or amounts or present in a tissue or a cell or populations thereof. A control can also be a cellular or tissue control, for example a tissue from a non-diseased state. A difference between a test sample and a control can be an increase or conversely a decrease. The difference can be a qualitative difference or a quantitative difference, for example a statistically significant difference.

Complementary: A double-stranded DNA or RNA strand consists of two complementary strands of base pairs. Complementary binding occurs when the base of one nucleic acid molecule forms a hydrogen bond to the base of another nucleic acid molecule. Normally, the base adenine (A) is complementary to thymidine (T) and uracil (U), while cytosine (C) is complementary to guanine (G). For example, the sequence 5′-ATCG-3′ of one ssDNA molecule can bond to 3′-TAGC-5′ of another ssDNA to form a dsDNA. In this example, the sequence 5′-ATCG-3′ is the reverse complement of 3′-TAGC-5′.

Nucleic acid molecules can be complementary to each other even without complete hydrogen-bonding of all bases of each molecule. For example, hybridization with a complementary nucleic acid sequence can occur under conditions of differing stringency in which a complement will bind at some but not all nucleotide positions.

Covalently linked: Refers to a covalent linkage between atoms by the formation of a covalent bond characterized by the sharing of pairs of electrons between atoms. In one example, a covalent link is a bond between an oxygen and a phosphorous, such as phosphodiester bonds in the backbone of a nucleic acid strand. In another example, a covalent link is one between a nucleic acid and a protein and/or nucleic acid that has been cross-linked to the nucleic acid by chemical means.

Cross-linking agent: A chemical agent or even light, that facilitates the attachment of one molecule to another molecule. Cross-linking agents can be protein-nucleic acid cross-linking agents, nucleic acid-nucleic acid cross-linking agents, and/or protein-protein cross-linking agents. Examples of such agents are known in the art. In some embodiments, a cross-linking agent is a reversible cross-linking agent. In some embodiments, a cross-linking agent is a non-reversible cross-linking agent.

Detectable label: A compound or composition that is conjugated directly or indirectly to another molecule to facilitate detection of that molecule. Specific, non-limiting examples of labels include fluorescent tags, enzymatic linkages, and radioactive isotopes. In some examples, a label is attached to an antibody or nucleic acid to facilitate detection of the molecule antibody or nucleic acid specifically binds.

DNA sequencing: The process of determining the nucleotide order of a given DNA molecule. Generally, the sequencing can be performed using automated Sanger sequencing (AB13730x1 genome analyzer), pyrosequencing on a solid support (454 sequencing, Roche), sequencing-by-synthesis with reversible terminations (ILLUMINA® Genome Analyzer), sequencing-by-ligation (ABI SOLiD®) or sequencing-by-synthesis with virtual terminators (HELISCOPE®).

In some embodiments, DNA sequencing is performed using a chain termination method developed by Frederick Sanger, and thus termed “Sanger based sequencing” or “SBS.” This technique uses sequence-specific termination of a DNA synthesis reaction using modified nucleotide substrates. Extension is initiated at a specific site on the template DNA by using a short oligonucleotide primer complementary to the template at that region. The oligonucleotide primer is extended using DNA polymerase in the presence of the four deoxynucleotide bases (DNA building blocks), along with a low concentration of a chain terminating nucleotide (most commonly a di-deoxynucleotide). Limited incorporation of the chain terminating nucleotide by the DNA polymerase results in a series of related DNA fragments that are terminated only at positions where that particular nucleotide is present. The fragments are then size-separated by electrophoresis a polyacrylamide gel, or in a narrow glass tube (capillary) filled with a viscous polymer. An alternative to using a labeled primer is to use labeled terminators instead; this method is commonly called “dye terminator sequencing.”

“Pyrosequencing” is an array-based method, which has been commercialized by 454 Life Sciences. In some embodiments of the array-based methods, single-stranded DNA is annealed to beads and amplified via EmPCR®. These DNA-bound beads are then placed into wells on a fiber-optic chip along with enzymes that produce light in the presence of ATP. When free nucleotides are washed over this chip, light is produced as the PCR amplification occurs and ATP is generated when nucleotides join with their complementary base pairs. Addition of one (or more) nucleotide(s) results in a reaction that generates a light signal that is recorded, such as by the charge coupled device (CCD) camera, within the instrument. The signal strength is proportional to the number of nucleotides, for example, homopolymer stretches, incorporated in a single nucleotide flow.

High throughput technique: Through a combination of robotics, data processing and control software, liquid handling devices, and detectors, high throughput techniques allows the rapid screening of potential reagents, conditions, or targets in a short period of time, for example in less than 24, less than 12, less than 6 hours, or even less than 1 hour.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide (or it's analog) and the DNA, or RNA. The oligonucleotide or oligonucleotide analog need not be 100% complementary to its target sequence to be specifically hybridizable. An oligonucleotide or analog is specifically hybridizable when there is a sufficient degree of complementarity to avoid non-specific binding of the oligonucleotide or analog to non-target sequences under conditions where specific binding is desired. Such binding is referred to as specific hybridization.

Isolated: An “isolated” biological component has been substantially separated or purified away from other biological components in the cell of the organism in which the component naturally occurs, for example, extra-chromatin DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids. It is understood that the term “isolated” does not imply that the biological component is free of trace contamination, and can include nucleic acid molecules that are at least 50% isolated, such as at least 75%, 80%, 90%, 95%, 98%, 99%, or even 100% isolated.

Nucleic acid (molecule or sequence): A deoxyribonucleotide or ribonucleotide polymer including without limitation, cDNA, mRNA, genomic DNA, and synthetic (such as chemically synthesized) DNA or RNA or hybrids thereof. The nucleic acid can be double-stranded (ds) or single-stranded (ss). Where single-stranded, the nucleic acid can be the sense strand or the antisense strand. Nucleic acids can include natural nucleotides (such as A, T/U, C, and G), and can also include analogs of natural nucleotides, such as labeled nucleotides. Some examples of nucleic acids include the probes disclosed herein.

The major nucleotides of DNA are deoxyadenosine 5 ‘-triphosphate (dATP or A), deoxyguanosine 5’-triphosphate (dGTP or G), deoxycytidine 5 ‘-triphosphate (dCTP or C) and deoxythymidine 5’-triphosphate (dTTP or T). The major nucleotides of RNA are adenosine 5 ‘-triphosphate (ATP or A), guanosine 5’-triphosphate (GTP or G), cytidine 5 ‘-triphosphate (CTP or C) and uridine 5’-triphosphate (UTP or U). Nucleotides include those nucleotides containing modified bases, modified sugar moieties, and modified phosphate backbones, for example as described in U.S. Pat. No. 5,866,336 to Nazarenko et al.

Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N˜6-sopentenyladenine, 1-methylguanine, 1-methylinosine, 2,2-dimethylguanine, 2-methyladenine, 2-methylguanine, 3-methylcytosine, 5-methyl cytosine, N6-adenine, 7-methylguanine, 5-methylaminomethyluracil, methoxyarninomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, 2,6-diaminopurine and biotinylated analogs, amongst others.

Examples of modified sugar moieties which may be used to modify nucleotides at any position on its structure include, but are not limited to arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

Peptide/Protein/Polypeptide: All of these terms refer to a polymer of amino acids and/or amino acid analogs that are joined by peptide bonds or peptide bond mimetics. The twenty naturally occurring amino acids and their single-letter and three-letter designations known in the art.

Sample: A sample, such as a biological sample, that includes biological materials (such as nucleic acids) obtained from an organism or a part thereof, such as a plant, or animal, and the like. In particular embodiments, the biological sample is obtained from an animal subject, such as a human subject. A biological sample is any solid or fluid sample obtained from, excreted by or secreted by any living organism, including without limitation, single celled organisms, such as bacteria, yeast, protozoans, and amebas among others, multicellular organisms (such as plants or animals, including samples from a healthy or apparently healthy human subject or a human patient affected by a condition or disease to be diagnosed or investigated). For example, a biological sample can be bone marrow, tissue biopsies, whole blood, serum, plasma, blood cells, endothelial cells, circulating tumor cells, lymphatic fluid, ascites fluid, interstitial fluid (also known as “extracellular fluid” and encompasses the fluid found in spaces between cells, including, inter alia, gingival crevicular fluid), cerebrospinal fluid (CSF), saliva, mucous, sputum, sweat, urine, or any other secretion, excretion, or other bodily fluids.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (i.e., 15±20*100=75).

One indication that two nucleic acid molecules are closely related is that the two molecules hybridize to each other under stringent conditions. Stringent conditions are sequence-dependent and are different under different environmental parameters.

Specific Binding Agent: An agent that binds substantially or preferentially only to a defined target such as a protein, enzyme, polysaccharide, oligonucleotide, DNA, RNA, recombinant vector or a small molecule.

A nucleic acid-specific binding agent binds substantially only to the defined nucleic acid, such as RNA, or to a specific region within the nucleic acid. In some embodiments a specific binding agent is a probe or primer, that specifically binds to a target nucleic acid of interest.

A protein-specific binding agent binds substantially only the defined protein, or to a specific region within the protein. For example, a “specific binding agent” includes antibodies and other agents that bind substantially to a specified polypeptide. Antibodies can be monoclonal or polyclonal antibodies that are specific for the polypeptide, as well as immunologically effective portions (“fragments”) thereof. The determination that a particular agent binds substantially only to a specific polypeptide may readily be made by using or adapting routine procedures. One suitable in vitro assay makes use of the Western blotting procedure (described in many standard texts, including Harlow and Lane, Using Antibodies: A Laboratory Manual, CSHL, New York, 1999).

Transposome: A transposase-transposon complexes. A conventional way for transposon mutagenesis usually place the transposase on the plasmid. In some such systems, termed “transposomes”, the transposase can form a functional complex with a transposon recognition site that is capable of catalyzing a transposition reaction. The transposase or integrase may bind to the transposase recognition site and insert the transposase recognition site into a target nucleic acid in a process sometimes termed “tagmentation”.

Transcription factor: A protein that regulates transcription. In particular, transcription factors regulate the binding of RNA polymerase and the initiation of transcription. A transcription factor binds upstream or downstream to either enhance or repress transcription of a gene by assisting or blocking RNA polymerase binding. The term transcription factor includes both inactive and activated transcription factors.

Transcription factors are typically modular proteins that affect regulation of gene expression. Exemplary transcription factors include but are not limited to AAF, ab1, ADA2, ADA-NF1, AF-1, AFP1, AhR, AIIN3, ALL-1, alpha-CBF, alpha-CP1, alpha-CP2a, alpha-CP2b, alphaHo, alphaH2-alphaH3, Alx-4, aMEF-2, AML1, AML1a, AML1b, AML1c, AML1DeltaN, AML2, AML3, AML3a, AML3b, AMY-1L, A-Myb, ANF, AP-1, AP-2alphaA, AP-2alphaB, AP-2beta, AP-2gamma, AP-3 (1), AP-3 (2), AP-4, AP-5, APC, AR, AREB6, Arnt, Arnt (774 M form), ARP-1, ATBF1-A, ATBF1-B, ATF, ATF-1, ATF-2, ATF-3, ATF-3deltaZIP, ATF-a, ATF-adelta, ATPF1, Barhl1, Barhl2, Barx1, Barx2, Bcl-3, BCL-6, BD73, beta-catenin, Bin1, B-Myb, BP1, BP2, brahma, BRCA1, Brn-3a, Brn-3b, Brn-4, BTEB, BTEB2, B-TFIID, C/EBPalpha, C/EBPbeta, C/EBPdelta, CACCbinding factor, Cart-1, CBF (4), CBF (5), CBP, CCAAT-binding factor, CCMT-binding factor, CCF, CCG1, CCK-1a, CCK-1b, CD28RC, cdk2, cdk9, Cdx-1, CDX2, Cdx-4, CFF, Chx10, CLIM1, CLIM2, CNBP, CoS, COUP, CP1, CP1A, CP1C, CP2, CPBP, CPE binding protein, CREB, CREB-2, CRE-BP1, CRE-BPa, CREMalpha, CRF, Crx, CSBP-1, CTCF, CTF, CTF-1, CTF-2, CTF-3, CTF-5, CTF-7, CUP, CUTL1, Cx, cyclin A, cyclin T1, cyclin T2, cyclin T2a, cyclin T2b, DAP, DAX1, DB1, DBF4, DBP, DbpA, DbpAv, DbpB, DDB, DDB-1, DDB-2, DEF, deltaCREB, deltaMax, DF-1, DF-2, DF-3, Dlx-1, Dlx-2, Dlx-3, DIx4 (long isoform), Dlx-4 (short isoform, Dlx-5, Dlx-6, DP-1, DP-2, DSIF, DSIF-p14, DSIF-p160, DTF, DUX1, DUX2, DUX3, DUX4, E, E12, E2F, E2F+E4, E2F+p107, E2F-1, E2F-2, E2F-3, E2F-4, E2F-5, E2F-6, E47, E4BP4, E4F, E4F1, E4TF2, EAR2, EBP-80, EC2, EF1, EF-C, EGR1, EGR2, EGR3, EIIaE-A, EIIaE-B, EIIaE-Calpha, EIIaE-Cbeta, EivF, EIf-1, EIk-1, Emx-1, Emx-2, Emx-2, En-1, En-2, ENH-bind. prot., ENKTF-1, EPAS1, epsilonF1, ER, Erg-1, Erg-2, ERR1, ERR2, ETF, Ets-1, Ets-1 deltaVil, Ets-2, Evx-1, F2F, factor 2, Factor name, FBP, f-EBP, FKBP59, FKHL18, FKHRL1P2, Fli-1, Fos, FOXB1, FOXC1, FOXC2, FOXD1, FOXD2, FOXD3, FOXD4, FOXE1, FOXE3, FOXF1, FOXF2, FOXG1a, FOXG1b, FOXG1c, FOXH1, FOXI1, FOXJ1a, FOXJ1b, FOXJ2 (long isoform), FOXJ2 (short isoform), FOXJ3, FOXK1a, FOXK1b, FOXK1c, FOXL1, FOXM1a, FOXM1b, FOXM1c, FOXN1, FOXN2, FOXN3, FOX01a, FOX01b, FOXO2, FOXO3a, FOXO3b, FOXO4, FOXP1, FOXP3, Fra-1, Fra-2, FTF, FTS, G factor, G6 factor, GABP, GABP-alpha, GABP-beta1, GABP-beta2, GADD 153, GAF, gammaCMT, gammaCAC1, gammaCAC2, GATA-1, GATA-2, GATA-3, GATA-4, GATA-5, GATA-6, Gbx-1, Gbx-2, GCF, GCMa, GCNS, GF1, GLI, GLI3, GR alpha, GR beta, GRF-1, Gsc, Gsc1, GT-IC, GT-IIA, GT-IIBalpha, GT-IIBbeta, H1TF1, H1TF2, H2RIIBP, H4TF-1, H4TF-2, HAND1, HAND2, HB9, HDAC1, HDAC2, HDAC3, hDaxx, heat-induced factor, HEB, HEB1-p67, HEB1-p94, HEF-1 B, HEF-1T, HEF-4C, HEN1, HEN2, Hesx1, Hex, HIF-1, HIF-1alpha, HIF-1beta, HiNF-A, HiNF-B, HINF-C, HINF-D, HiNF-D3, HiNF-E, HiNF-P, HIP1, HIV-EP2, Hlf, HLTF, HLTF (Met123), HLX, HMBP, HMG I, HMG I(Y), HMG Y, HMGI-C, HNF-1A, HNF-1B, HNF-1C, HNF-3, HNF-3alpha, HNF-3beta, HNF-3gamma, HNF4, HNF-4alpha, HNF4alpha1, HNF-4alpha2, HNF-4alpha3, HNF-4alpha4, HNF4gamma, HNF-6alpha, hnRNP K, HOX11, HOXA1, HOXA10, HOXA10 PL2, HOXA11, HOXA13, HOXA2, HOXA3, HOXA4, HOXA5, HOXA6, HOXA7, HOXA9A, HOXA9B, HOXB-1, HOXB13, HOXB2, HOXB3, HOXB4, HOXBS, HOXB6, HOXA5, HOXB7, HOXB8, HOXB9, HOXC10, HOXC11, HOXC12, HOXC13, HOXC4, HOXC5, HOXC6, HOXC8, HOXC9, HOXD10, HOXD11, HOXD12, HOXD13, HOXD3, HOXD4, HOXD8, HOXD9, Hp55, Hp65, HPX42B, HrpF, HSF, HSF1 (long), HSF1 (short), HSF2, hsp56, Hsp90, IBP-1, ICER-II, ICER-ligamma, ICSBP, Id1, Id1 H′, Id2, Id3, Id3/Heir-1, IF1, IgPE-1, IgPE-2, IgPE-3, IkappaB, IkappaB-alpha, IkappaB-beta, IkappaBR, II-1 RF, IL-6 RE-BP, 11-6 RF, INSAF, IPF1, IRF-1, IRF-2, irlB, IRX2a, Irx-3, Irx-4, ISGF-1, ISGF-3, ISGF3alpha, ISGF-3gamma, 1st-1, ITF, ITF-1, ITF-2, JRF, Jun, JunB, JunD, kappay factor, KBP-1, KER1, KER-1, Kox1, KRF-1, Ku autoantigen, KUP, LBP-1, LBP-1a, LBX1, LCR-F1, LEF-1, LEF-1B, LF-A1, LHX1, LHX2, LHX3a, LHX3b, LHXS, LHX6.1a, LHX6.1b, LIT-1, Lmo1, Lmo2, LMX1A, LMX1B, L-My1 (long form), L-My1 (short form), L-My2, LSF, LXRalpha, LyF-1, LyI-1, M factor, Mad1, MASH-1, Max1, Max2, MAZ, MAZ1, MB67, MBF1, MBF2, MBF3, MBP-1 (1), MBP-1 (2), MBP-2, MDBP, MEF-2, MEF-2B, MEF-2C (433 AA form), MEF-2C (465 AA form), MEF-2C (473 M form), MEF-2C/delta32 (441 AA form), MEF-2D00, MEF-2D0B, MEF-2DA0, MEF-2DA′0, MEF-2DAB, MEF-2DA′B, Meis-1, Meis-2a, Meis-2b, Meis-2c, Meis-2d, Meis-2e, Meis3, Meox1, Meox1a, Meox2, MHox (K-2), Mi, MIF-1, Miz-1, MM-1, MOP3, MR, Msx-1, Msx-2, MTB-Zf, MTF-1, mtTF1, Mxi1, Myb, Myc, Myc 1, Myf-3, Myf-4, Myf-5, Myf-6, MyoD, MZF-1, NC1, NC2, NCX, NELF, NER1, Net, NF III-a, NF NF NF-1, NF-1A, NF-1B, NF-1X, NF-4FA, NF-4FB, NF-4FC, NF-A, NF-AB, NFAT-1, NF-AT3, NF-Atc, NF-Atp, NF-Atx, NfbetaA, NF-CLE0a, NF-CLE0b, NFdeltaE3A, NFdeltaE3B, NFdeltaE3C, NFdeltaE4A, NFdeltaE4B, NFdeltaE4C, Nfe, NF-E, NF-E2, NF-E2 p45, NF-E3, NFE-6, NF-Gma, NF-GMb, NF-IL-2A, NF-IL-2B, NF-jun, NF-kappaB, NF-kappaB(-like), NF-kappaB1, NF-kappaB1, precursor, NF-kappaB2, NF-kappaB2 (p49), NF-kappaB2 precursor, NF-kappaE1, NF-kappaE2, NF-kappaE3, NF-MHCIIA, NF-MHCIIB, NF-muE1, NF-muE2, NF-muE3, NF-S, NF-X, NF-X1, NF-X2, NF-X3, NF-Xc, NF-YA, NF-Zc, NF-Zz, NHP-1, NHP-2, NHP3, NHP4, NKX2-5, NKX2B, NKX2C, NKX2G, NKX3A, NKX3A v1, NKX3A v2, NKX3A v3, NKX3A v4, NKX3B, NKX6A, Nmi, N-Myc, N-Oct-2alpha, N-Oct-2beta, N-Oct-3, N-Oct-4, N-Oct-5a, N-Oct-5b, NP-TCII, NR2E3, NR4A2, Nrf1, Nrf-1, Nrf2, NRF-2beta1, NRF-2gamma1, NRL, NRSF form 1, NRSF form 2, NTF, 02, OCA-B, Oct-1, Oct-2, Oct-2.1, Oct-2B, Oct-2C, Oct-4A, Oct4B, Oct-5, Oct-6, Octa-factor, octamer-binding factor, oct-B2, oct-B3, Otx1, Otx2, OZF, p107, p130, p28 modulator, p300, p38erg, p45, p49erg,-p53, p55, p55erg, p65delta, p67, Pax-1, Pax-2, Pax-3, Pax-3A, Pax-3B, Pax-4, Pax-5, Pax-6, Pax-6/Pd-5a, Pax-7, Pax-8, Pax-8a, Pax-8b, Pax-8c, Pax-8d, Pax-8e, Pax-8f, Pax-9, Pbx-1a, Pbx-1b, Pbx-2, Pbx-3a, Pbx-3b, PC2, PC4, PC5, PEA3, PEBP2alpha, PEBP2beta, Pit-1, PITX1, PITX2, PITX3, PKNOX1, PLZF, PO-B, Pontin52, PPARalpha, PPARbeta, PPARgamma1, PPARgamma2, PPUR, PR, PR A, pRb, PRD1-BF1, PRDI-BFc, Prop-1, PSE1, P-TEFb, PTF, PTFalpha, PTFbeta, PTFdelta, PTFgamma, Pu box binding factor, Pu box binding factor (BJA-B), PU.1, PuF, Pur factor, R1, R2, RAR-alpha1, RAR-beta, RAR-beta2, RAR-gamma, RAR-gamma1, RBP60, RBP-Jkappa, Rel, RelA, RelB, RFX, RFX1, RFX2, RFX3, RFXS, RF-Y, RORalpha1, RORalpha2, RORalpha3, RORbeta, RORgamma, Rox, RPF1, RPGalpha, RREB-1, RSRFC4, RSRFC9, RVF, RXR-alpha, RXR-beta, SAP-1a, SAP1b, SF-1, SHOX2a, SHOX2b, SHOXa, SHOXb, SHP, SIII-p110, SIII-p15, SIII-p18, SIM′, Six-1, Six-2, Six-3, Six-4, Six-5, Six-6, SMAD-1, SMAD-2, SMAD-3, SMAD-4, SMAD-5, SOX-11, SOX-12, Sox-4, Sox-5, SOX-9, Sp1, Sp2, Sp3, Sp4, Sph factor, Spi-B, SPIN, SRCAP, SREBP-1a, SREBP-1b, SREBP-1c, SREBP-2, SRE-ZBP, SRF, SRY, SRP1, Staf-50, STAT1alpha, STAT1beta, STAT2, STAT3, STAT4, STATE, T3R, T3R-alpha1, T3R-alpha2, T3R-beta, TAF(I)110, TAF(I)48, TAF(I)63, TAF(II)100, TAF(II)125, TAF(II)135, TAF(II)170, TAF(II)18, TAF(II)20, TAF(II)250, TAF(II)250Delta, TAF(II)28, TAF(II)30, TAF(II)31, TAF(II)55, TAF(II)70-alpha, TAF(II)70-beta, TAF(II)70-gamma, TAF-I, TAF-II, TAF-L, Tal-1, Tal-1beta, Tal-2, TAR factor, TBP, TBX1A, TBX1B, TBX2, TBX4, TBXS (long isoform), TBXS (short isoform), TCF, TCF-1, TCF-1A, TCF-1B, TCF-1C, TCF-1D, TCF-1E, TCF-1F, TCF-1G, TCF-2alpha, TCF-3, TCF-4, TCF-4(K), TCF-4B, TCF-4E, TCFbeta1, TEF-1, TEF-2, tel, TFE3, TFEB, TFIIA, TFIIA-alpha/beta precursor, TFIIA-alpha/beta precursor, TFIIA-gamma, TFIIB, TFIID, TFIIE, TFIIE-alpha, TFIIE-beta, TFIIF, TFIIF-alpha, TFIIF-beta, TFIIH, TFIIH*, TFIIH-CAK, TFIIH-cyclin H, TFIIH-ERCC2/CAK, TFIIH-MAT1, TFIIH-MO15, TFIIH-p34, TFIIH-p44, TFIIH-p62, TFIIH-p80, TFIIH-p90, TFII-I, Tf-LF1, Tf-LF2, TGIF, TGIF2, TGT3, THRA1, TIF2, TLE1, TLX3, TMF, TR2, TR2-11, TR2-9, TR3, TR4, TRAP, TREB-1, TREB-2, TREB-3, TREF1, TREF2, TRF (2), TTF-1, TXRE BP, TxREF, UBF, UBP-1, UEF-1, UEF-2, UEF-3, UEF-4, USF1, USF2, USF2b, Vav, Vax-2, VDR, vHNF-1A, vHNF-1B, vHNF-1C, VITF, WSTF, WT1, WT1I, WT1 I-KTS, WT1 I-del2, WT1-KTS, WT1-del2, X2BP, XBP-1, XW-V, XX, YAF2, YB-1, YEBP, YY1, ZEB, ZF1, ZF2, ZFX, ZHX1, ZIC2, ZID, ZNF174, amongst others.

An activated transcription factor is a transcription factor that has been activated by a stimulus resulting in a measurable change in the state of the transcription factor, for example a post-translational modification, such as phosphorylation, methylation, and the like. Activation of a transcription factor can result in a change in the affinity for a particular DNA sequence or of a particular protein, such as another transcription factor and/or cofactor.

Under conditions that permit binding: A phrase used to describe any environment that permits the desired activity, for example conditions under which two or more molecules, such as nucleic acid molecules and/or protein molecules, can bind. Such conditions can include specific concentrations of salts and/or other chemicals that facilitate the binding of molecules.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999. In addition, the materials, methods, and examples are illustrative only and not intended to be limiting

### II. Description of Several Embodiments

### A. Introduction

ChIP is a powerful method to selectively enrich for DNA sequences bound by a particular protein in living cells. However, the widespread use of this method has been limited by the lack of a sufficiently robust method to identify all of the enriched DNA sequences.

Sample preparation of ChIP DNA for next-generation sequencing can involve fragmentation of genomic DNA into smaller fragments, followed by addition of functional tag sequences (“tags”) to the strands of the fragments. Such tags include priming sites for DNA polymerases for sequencing reactions, restriction sites, and domains for capture, amplification, detection, address, and transcription promoters. Previous methods for generating DNA fragment libraries required fragmenting the target DNA mechanically using a sonicator, nebulizer, or by a nuclease, and then joining (e.g., by ligation) the oligonucleotides containing the tags to the ends of the fragments. During these steps, significant amounts of sample can be lost or degraded, imposing lower limits on the amount on input sample needed. Thus, it can be especially frustrating for the researcher, especially when working with primary samples obtained from a subject. Thus, additional methods of increasing the yield and quality of ChIP-Seq DNA for analysis are needed.

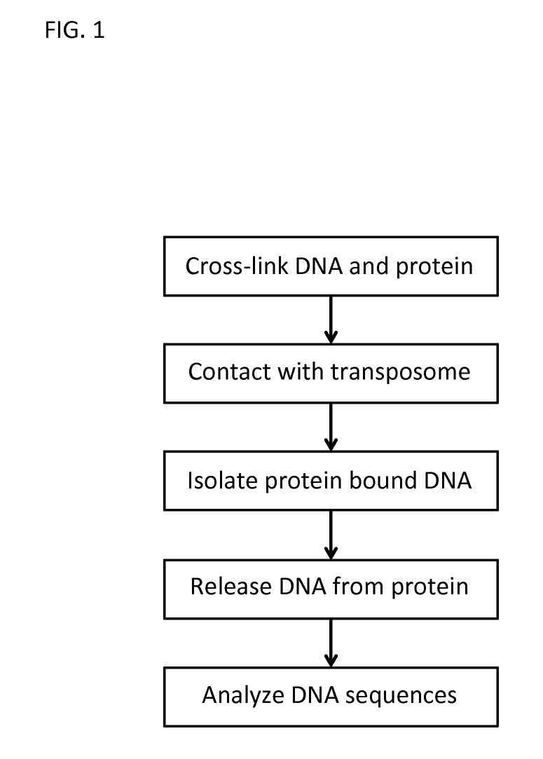

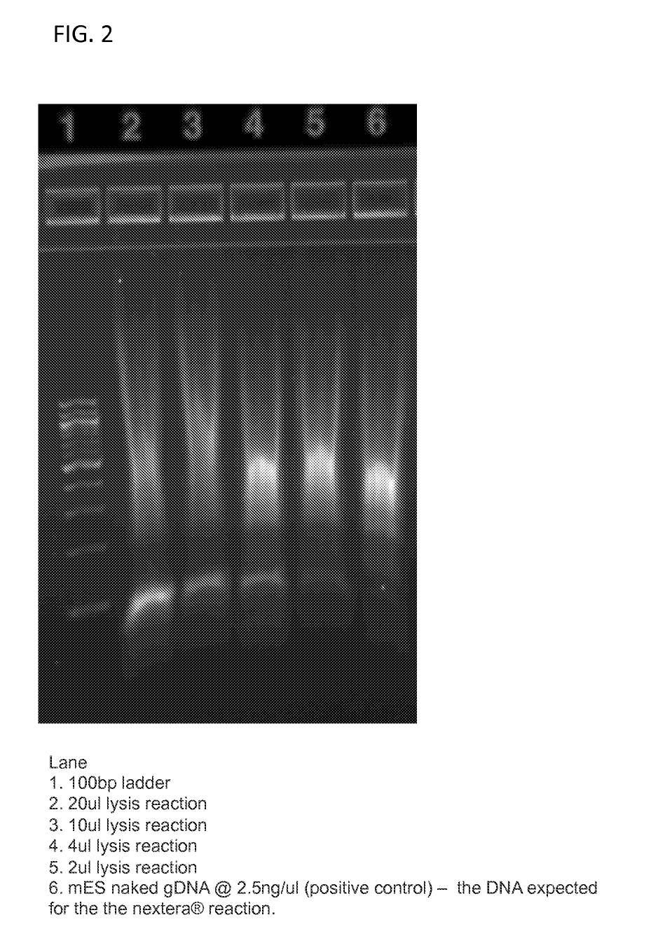

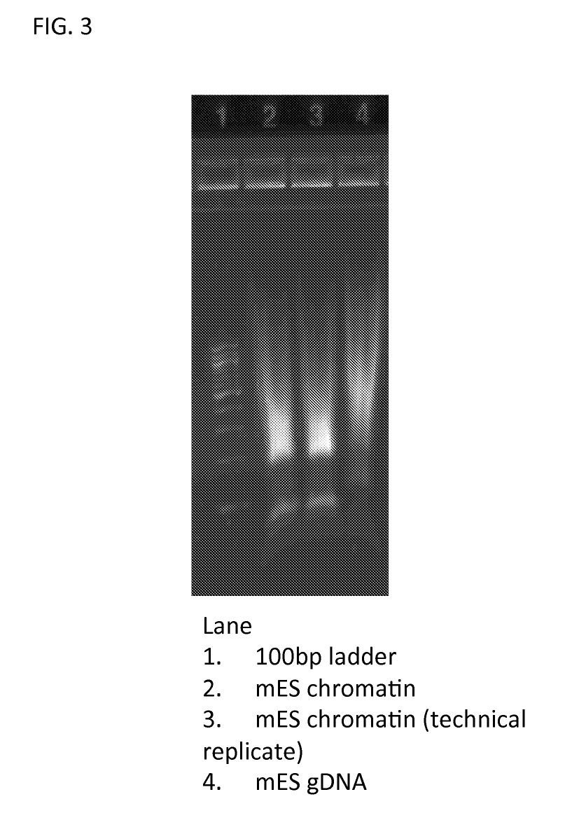

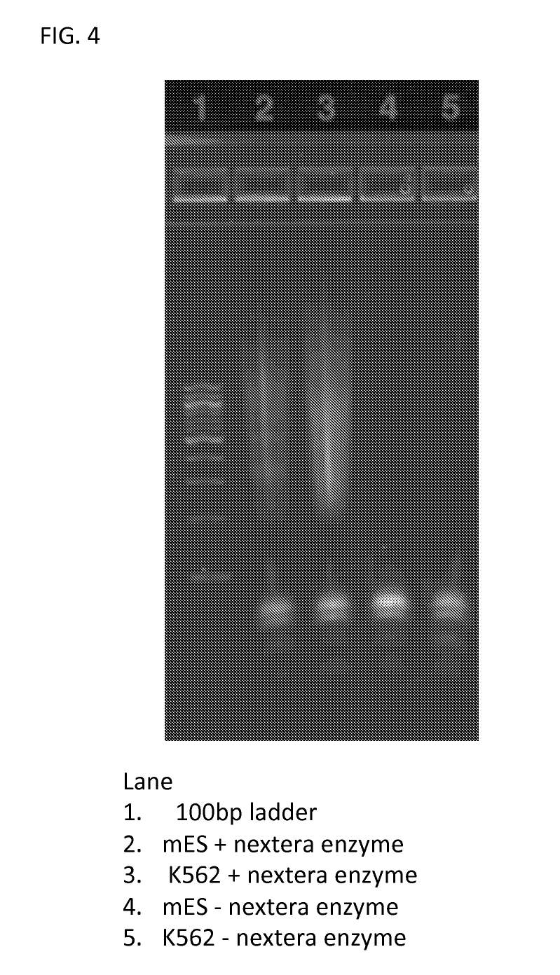

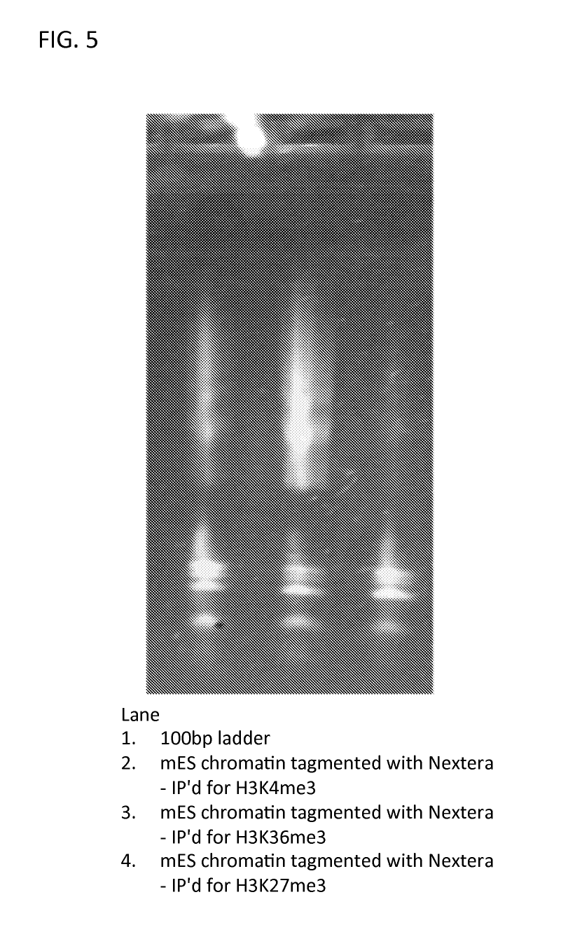

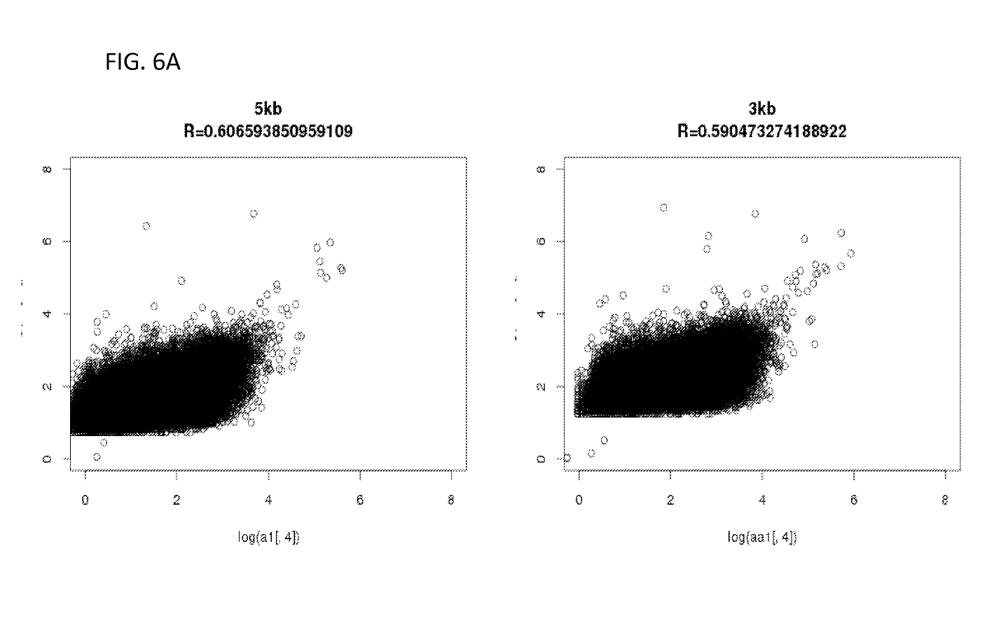

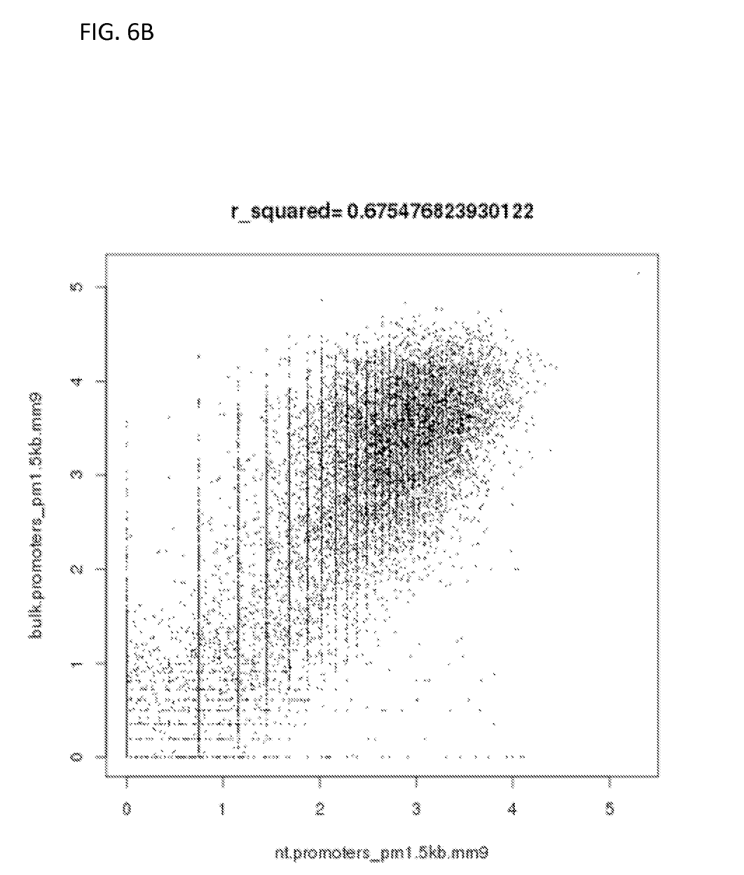

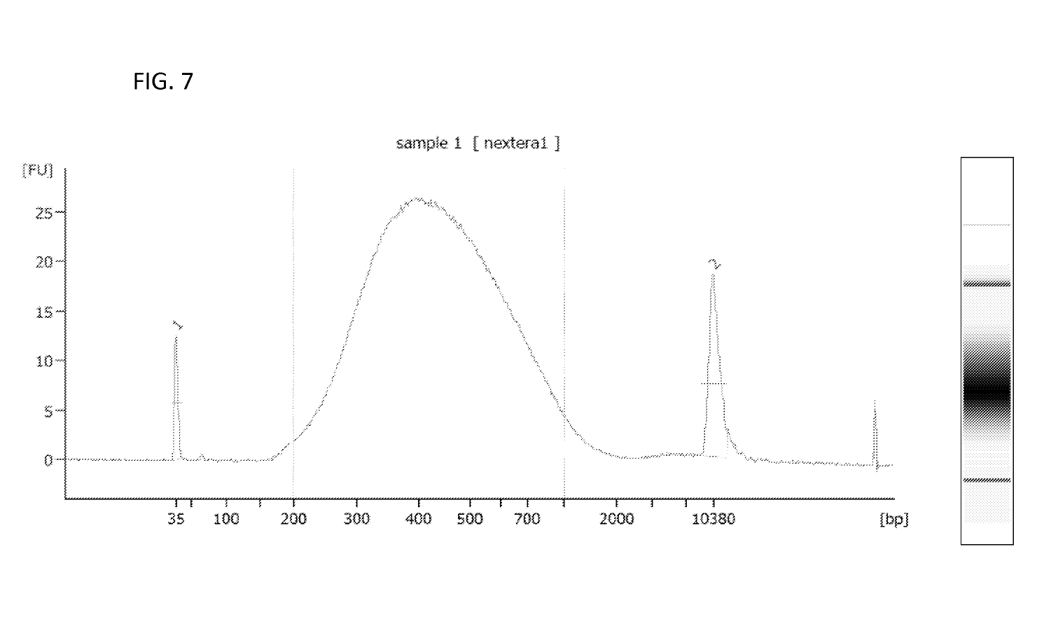

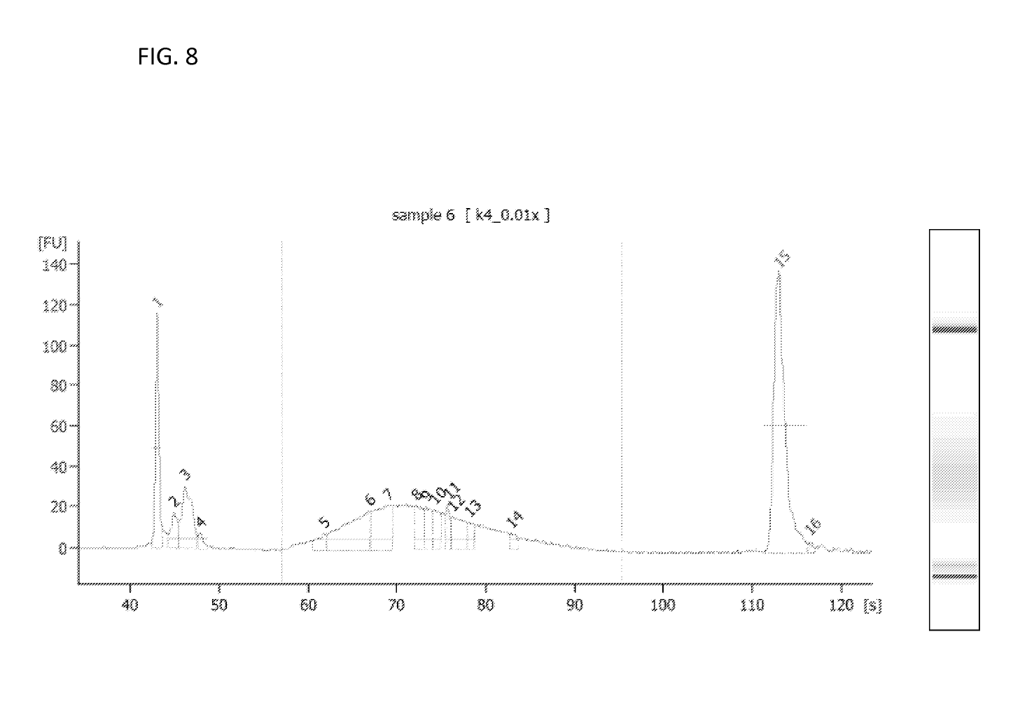

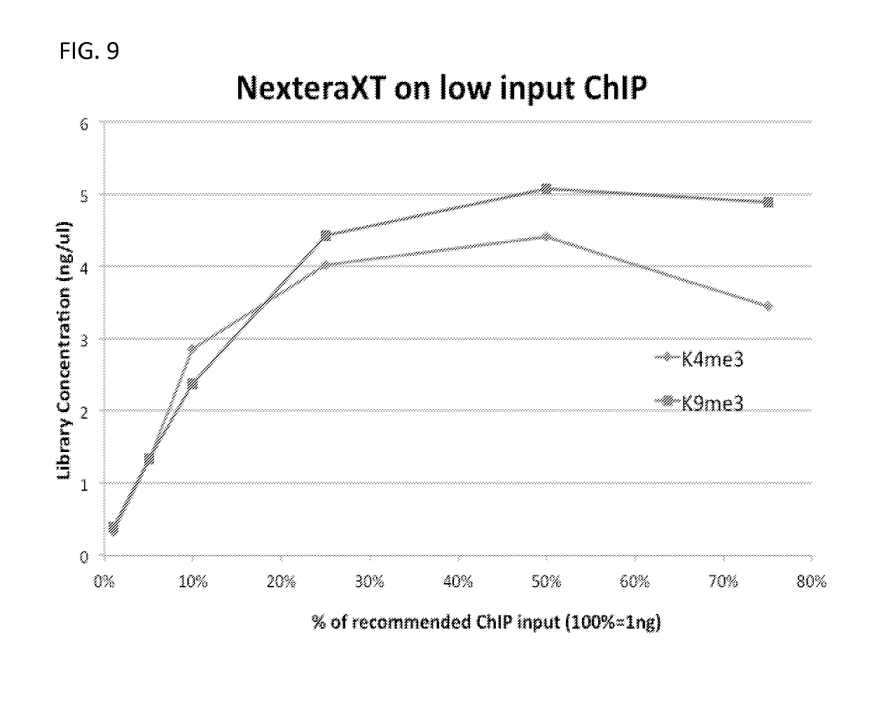

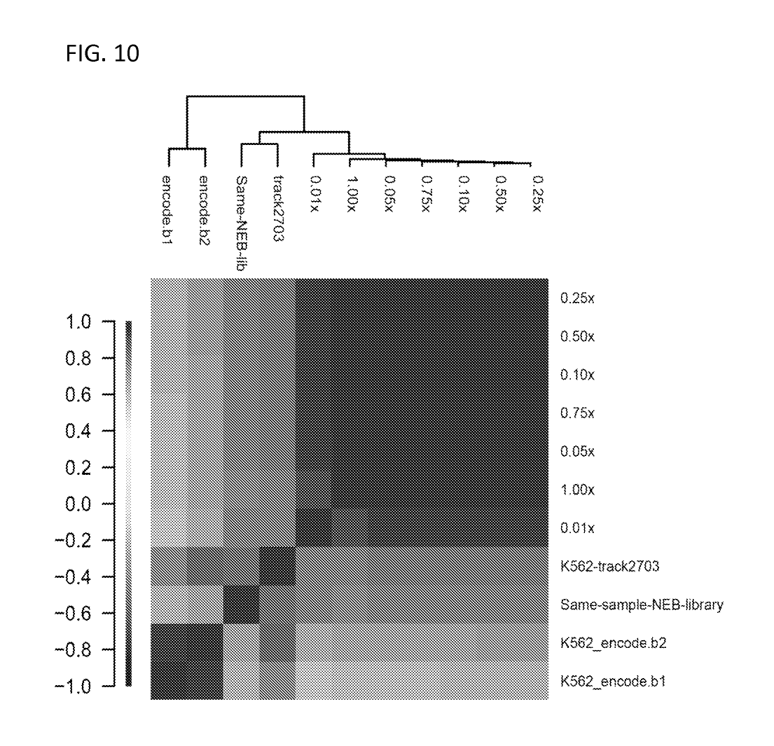

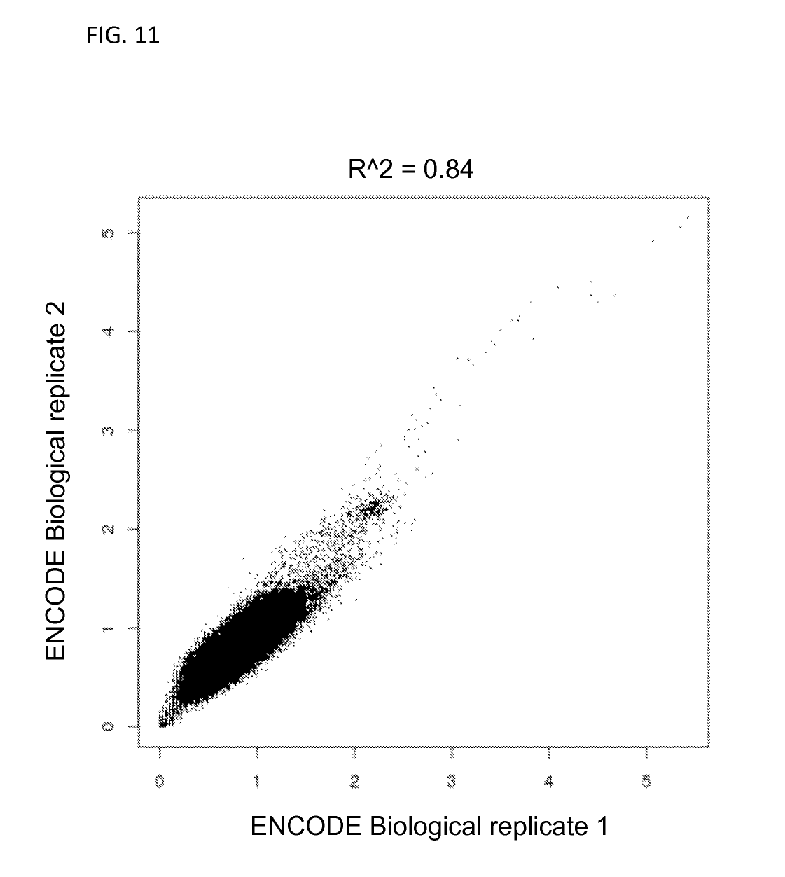

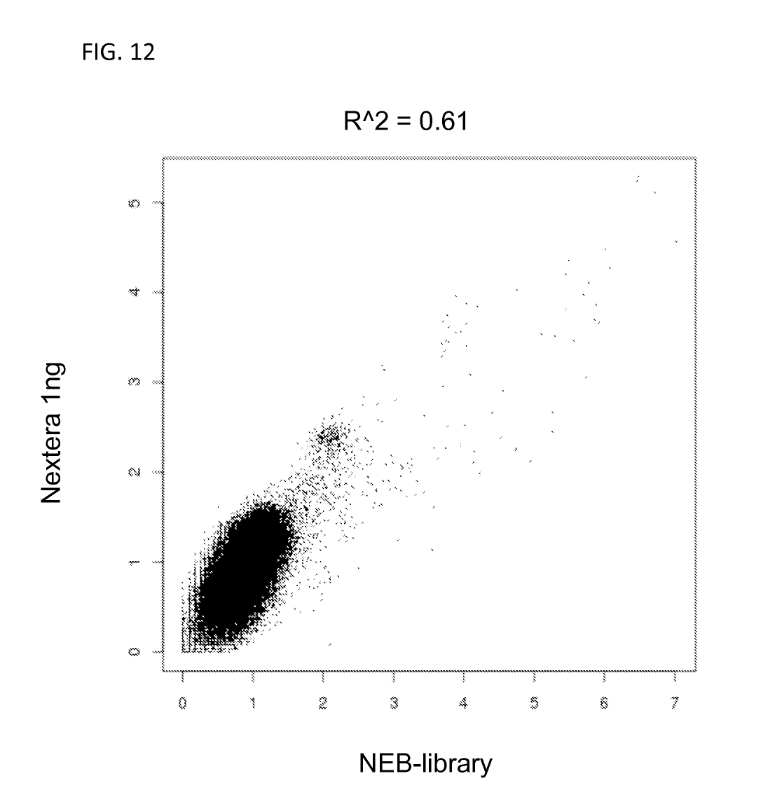

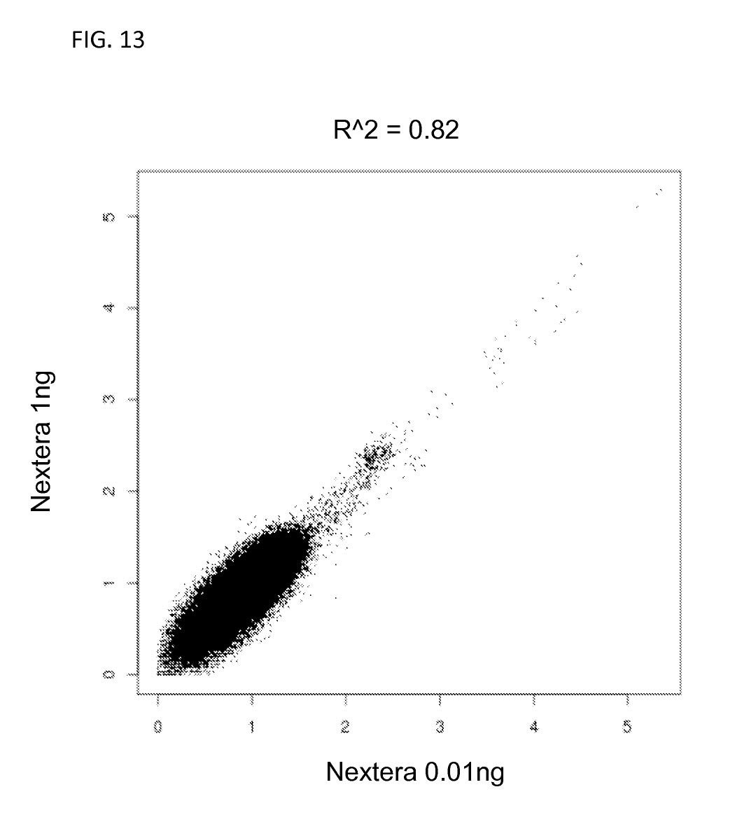

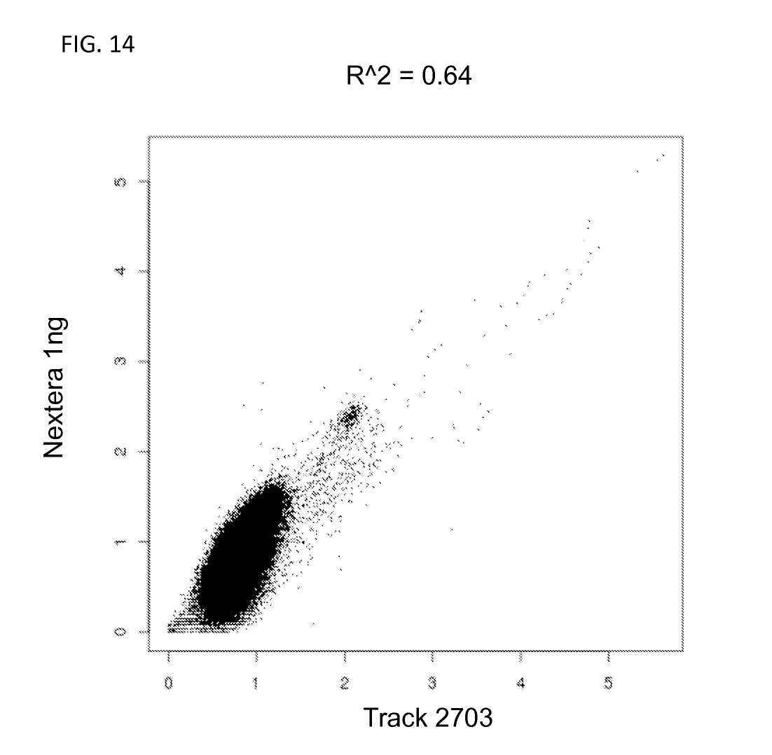

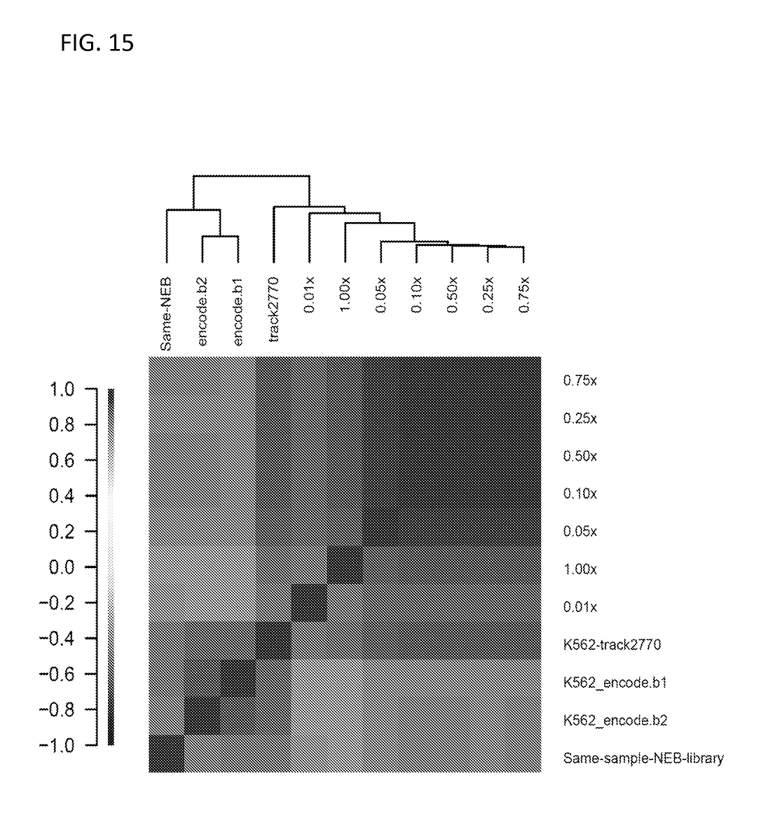

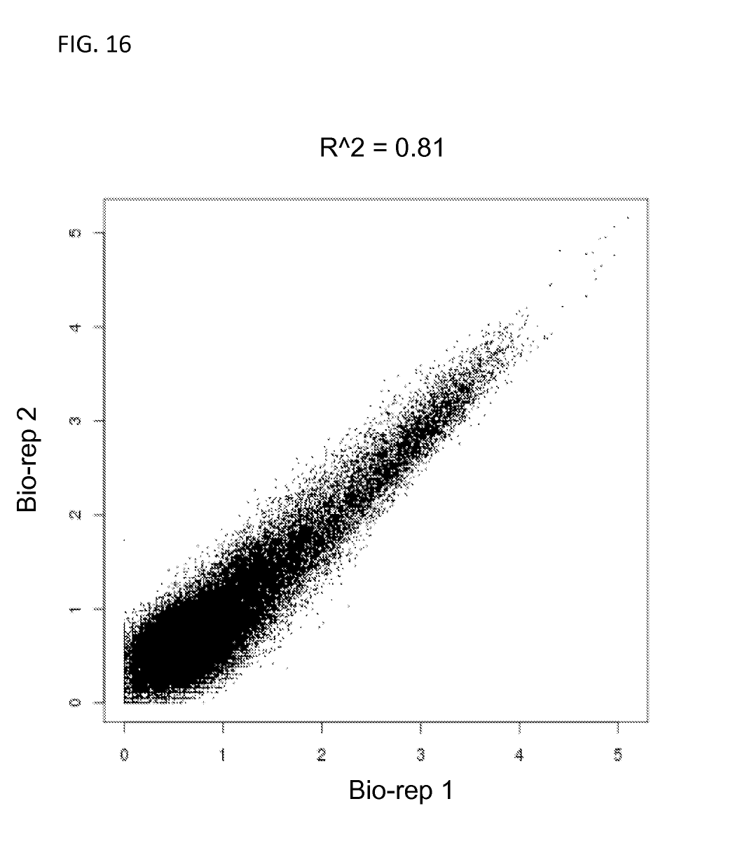

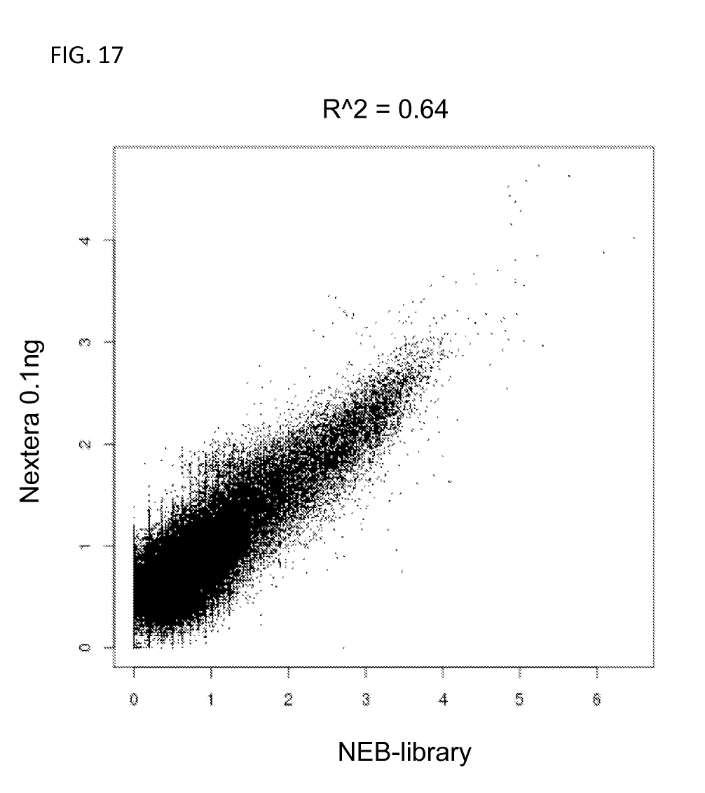

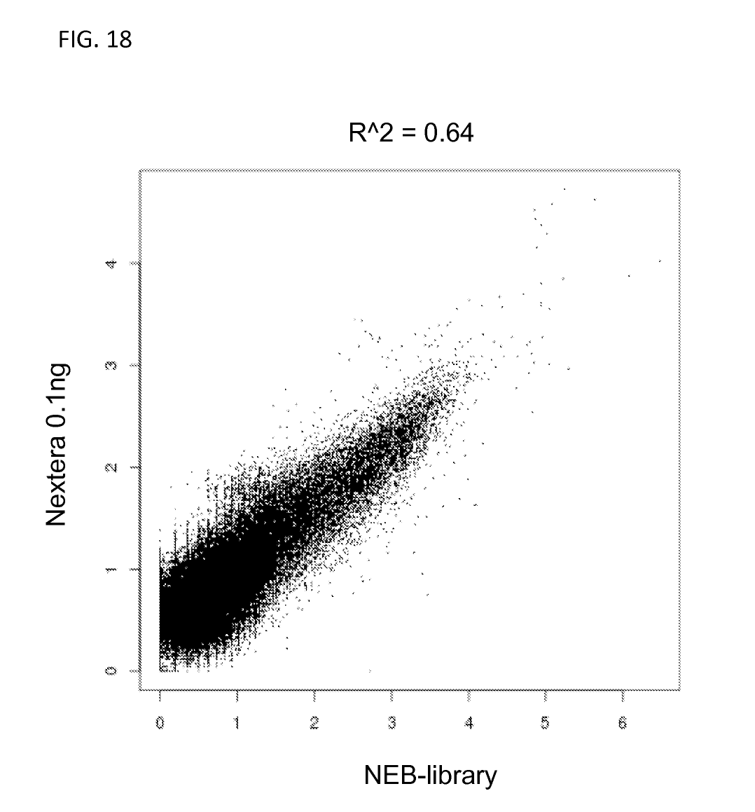

In order to improve the quality and the yield and quality of ChIP-Seq DNA for analysis, the inventors have developed a transposon shearing and tagging system for chromatin DNA. FIG. 1 is a flow chart showing an example method according to embodiments of the disclosed methods. The disclosed methods improve both the quality and yield of DNA for use in the sequencing steps, by completing the shearing and tagging step in a single reaction. In addition, to overcome possible bias toward introduction of tagmentation to open chromatin, the inventors have refined their technique to overcome such bias.

Thus provided herein, is a method of tagmentation of chromatin DNA that can be used in a high-throughput indexed method for systematic mapping of in vivo protein-DNA binding that greatly. The disclosed methods increase the throughput, while significantly reducing the labor and cost required for ChlP-Seq. The disclosed methods can be used to prepare a library of tagmented chromatin DNA molecules. In some embodiments, the methods overcome inherent bias of a transposome for open chromatin structures.

### B. Methods

Disclosed herein are methods for shearing and tagging chromatin DNA. The disclosed methods include contacting chromatin DNA, under conditions that permit integration of a transposon into chromatin DNA, with at least one artificial transposome. The artificial transposome includes at least one transposase and a transposon. The transposon includes a first DNA molecule comprising a first transposase recognition site and a second DNA molecule comprising a second transposase recognition site. Integration of the transposon (or really the two parts of the broken transposon) yields a sheared (or fragmented) DNA with the first and second DNA molecules integrated on either side of the fragmentation site. In this way, the chromatin DNA is both fragmented and tagged at the fragmentation site. In some examples, the transposase recognition sites have the same sequence, while in other examples, the transposase recognition sites have different sequences. With multiple insertions throughout the chromatin DNA, the DNA is effectively fragmented into small fragments amenable to analysis by next generation sequencing methods. In some embodiments, the chromatin DNA is contacted with at least two different transposomes, and wherein the different transposomes comprise different DNA sequences. Thus, the tagged chromatin DNA can be tagged at the 5′ and 3′ end with different transposon sequences. In some examples the first and second DNA molecules are connected, for example by one or more sites for a restriction enzyme, such that the transposon can be cut at a later time.

The first and second DNA molecules of the transposon can further include a variety of tag sequences, which can be added covalently to the fragments in the process of the disclosed method. As used herein, the term “tag” means a nucleotide sequence that is attached to another nucleic acid to provide the nucleic acid with some functionality. Examples of tags include barcodes, primer sites, affinity tags, and reporter moieties or any combination thereof.

In some embodiments, the first and/or second DNA molecule further include a barcode, which can be the same or different. These nucleic acid barcodes can be used to tag the fragmented DNA, for example by sample, organism, or the like, for example so that multiple samples can be analyzed simultaneously while preserving information about the sample origin. Generally, a barcode can include one or more nucleotide sequences that can be used to identify one or more particular nucleic acids. The barcode can be an artificial sequence, or can be a naturally occurring sequence. A barcode can comprise at least about 1, 2, 3, 4, 5, 6, 7, 8, 9, 10, 11, 12, 13, 14, 15, 16, 17, 18, 19, 20 or more consecutive nucleotides. In some embodiments, a barcode comprises at least about 10, 20, 30, 40, 50, 60, 70 80, 90, 100 or more consecutive nucleotides. In some embodiments, at least a portion of the barcodes in a population of nucleic acids comprising barcodes is different. In some embodiments, at least about 10%, 20%, 30%, 40%, 50%, 60%, 70%, 80%, 90%, 95%, 99% of the barcodes are different. In more such embodiments, all of the barcodes are different. The diversity of different barcodes in a population of nucleic acids comprising barcodes can be randomly generated or non-randomly generated. In some embodiments, a transposon sequence comprises at least one barcode. In some embodiments, a transposon sequence comprises a barcode comprising a first barcode sequence and a second barcode sequence. In some such embodiments, the first barcode sequence can be identified or designated to be paired with the second barcode sequence. For example, a known first barcode sequence can be known to be paired with a known second barcode sequence using a reference table comprising a plurality of first and second bar code sequences known to be paired to one another. In another example, the first barcode sequence can comprise the same sequence as the second barcode sequence. In another example, the first barcode sequence can comprise the reverse complement of the second barcode sequence. In some embodiments, the first barcode sequence and the second barcode sequence are different (“bi-codes”). It will be understood that in some embodiments, the vast number of available barcodes permits each tagmented nucleic acid molecule to comprise a unique identification. Unique identification of each molecule in a mixture of template nucleic acids can be used in several applications to identify individual nucleic acid molecules, in samples having multiple chromosomes, genomes, cells, cell types, cell disease states, and species, for example in haplotype sequencing, parental allele discrimination, metagenomic sequencing, and sample sequencing of a genome.

In some embodiments, the first and/or second DNA molecule includes a sequencing adaptor. The sequencing adaptors may be the same or different. The inclusion of a sequence adaptor facilitates the sequencing of the fragmented DNA produced, for example using next generation sequencing, such as array-based sequencing.

In some embodiments, the first and/or second DNA molecule includes a universal priming site. The universal priming site(s) may be the same or different. The inclusion of a universal priming site facilitates the amplification of the fragmented DNA produced, for example using PCR based amplification. The orientation of the primer sites in such embodiments can be such that a primer hybridizing to the first primer site and a primer hybridizing to the second primer site are in the same orientation, or in different orientations. In one embodiment, the primer sequence can be complementary to a primer used for amplification. In another embodiment, the primer sequence is complementary to a primer used for sequencing.

In some embodiments, a tag can be an affinity tag. Affinity tags can be useful for the bulk separation of target nucleic acids hybridized to hybridization tags. As used herein, the term “affinity tag” and grammatical equivalents can refer to a component of a multi-component complex, wherein the components of the multi-component complex specifically interact with or bind to each other. For example, an affinity tag can include biotin or His that can bind streptavidin or nickel, respectively. Other examples of multiple-component affinity tag complexes include, ligands and their receptors, for example, avidin-biotin, streptavidin-biotin, and derivatives of biotin, streptavidin, or avidin, including, but not limited to, 2-iminobiotin, desthiobiotin, NeutrAvidin, CaptAvidin, and the like; binding proteins/peptides, including maltose-maltose binding protein (MBP), calcium-calcium binding protein/peptide (CBP); antigen-antibody, including epitope tags, and their corresponding anti-epitope antibodies; haptens, for example, dinitrophenyl and digoxigenin, and their corresponding antibodies; aptamers and their corresponding targets; poly-His tags (e.g., penta-His and hexa-His) and their binding partners including corresponding immobilized metal ion affinity chromatography (IMAC) materials and anti-poly-His antibodies; fluorophores and anti-fluorophore antibodies; and the like.

In some embodiments, a tag can comprise a reporter moiety. As used herein, the term “reporter moiety” and grammatical equivalents can refer to any identifiable tag, label, or group. The skilled artisan will appreciate that many different species of reporter moieties can be used with the methods and compositions described herein, either individually or in combination with one or more different reporter moieties. In certain embodiments, a reporter moiety can emit a signal. Examples of signals are a fluorescent, a chemiluminescent, a bioluminescent, a phosphorescent, a radioactive, a calorimetric, or an electrochemiluminescent signals. Example reporter moieties include fluorophores, radioisotopes, chromogens, enzymes, antigens including epitope tags, semiconductor nanocrystals such as quantum dots, heavy metals, dyes, phosphorescence groups, chemiluminescent groups, electrochemical detection moieties, binding proteins, phosphors, rare earth chelates, transition metal chelates, near-infrared dyes, electrochemiluminescence labels, and mass spectrometer compatible reporter moieties, such as mass tags, charge tags, and isotopes. More reporter moieties that may be used with the methods and compositions described herein include spectral labels such as fluorescent dyes (e.g., fluorescein isothiocyanate, Texas red, rhodamine, and the like), radiolabels (e.g., H, I, S, C, P, ³³P, etc.), enzymes (e.g., horseradish peroxidase, alkaline phosphatase etc.) spectral calorimetric labels such as colloidal gold or colored glass or plastic (e.g. polystyrene, polypropylene, latex, etc.) beads; magnetic, electrical, thermal labels; and mass tags.

Reporter moieties can also include enzymes (horseradish peroxidase, etc.) and magnetic particles. More reporter moieties include chromophores, phosphors and fluorescent moieties, for example, Texas red, dixogenin, biotin, 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bis-benzoxazole, bis-p-oxazolyl benzene, 1,2-benzophenazin, retinol, bis-3-aminopyridinium salts, hellebrigenin, tetracycline, sterophenol, benzimidazolylphenylamine, 2-oxo-3-chromen, indole, xanthen, 7-hydroxycoumarin, phenoxazine, calicylate, strophanthidin, porphyrins, triarylmethanes and flavin. Individual fluorescent compounds which have functionalities for linking to an element desirably detected in an apparatus or assay provided herein, or which can be modified to incorporate such functionalities include, e.g., dansyl chloride; fluoresceins such as 3,6-dihydroxy-9-phenylxanthydrol; rhodamineisothiocyanate; N-phenyl 1-amino-8-sulfonatonaphthalene; N-phenyl 2-amino-6-sulfonatonaphthalene; 4-acetamido-4-isothiocyanato-stilbene-2,2′-disulfonic acid; pyrene-3-sulfonic acid; 2-toluidinonaphthalene-6-sulfonate; N-phenyl-N-methyl-2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine: N,N′-dihexyl oxacarbocyanine; merocyanine, 4-(3′-pyrenyl)stearate; d-3-aminodesoxy-equilenin; 12-(9′-anthroyl)stearate; 2-methylanthracene; 9-vinylanthracene; 2,2′(vinylene-p-phenylene)bisbenzoxazole; p-bis(2-methyl-5-phenyl-oxazolyl))benzene; 6-dimethylamino-1,2-benzophenazin; retinol; bis(3′-aminopyridinium) 1,10-decandiyl diiodide; sulfonaphthylhydrazone of hellibrienin; chlorotetracycline; N-(7-dimethylamino-4-methyl-2-oxo-3-chromenyl)maleimide; N-(p-(2benzimidazolyl)-phenyl)maleimide; N-(4-fluoranthyl)maleimide; bis(homovanillic acid); resazarin; 4-chloro7-nitro-2, 1,3-benzooxadiazole; merocyanine 540; resorufin; rose bengal; 2,4-diphenyl-3(2H)-furanone, fluorescent lanthanide complexes, including those of Europium and Terbium, fluorescein, rhodamine, tetramethylrhodamine, eosin, erythrosin, coumarin, methyl-coumarins, quantum dots (also referred to as “nanocrystals”: see U.S. Pat. No. 6,544,732), pyrene, Malachite green, stilbene, Lucifer Yellow, Cascade Blue™, Texas Red, Cy dyes (Cy3, Cy5, etc.), Alexa Fluor® dyes, phycoerythin, bodipy, and others described in the 6th Edition of the Molecular Probes Handbook by Richard P. Haugland.

The disclosed methods can use any transposase. Some embodiments can include the use of a hyperactive Tn5 transposase and a Tn5-type transposase recognition site (Goryshin and Reznikoff, J. Biol. Chem., 273:7367 (1998)), or MuA transposase and a Mu transposase recognition site comprising R1 and R2 end sequences (Mizuuchi, K., Cell, 35: 785, 1983; Savilahti, H, et al, EMBO J., 14: 4893, 1995). An exemplary transposase recognition site that forms a complex with a hyperactive Tn5 transposase (e.g., EZ-Tn5™ Transposase). More examples of transposition systems that can be used with certain embodiments provided herein include Staphylococcus aureus Tn552 (Colegio et al, J. Bacteriol, 183: 2384-8, 2001; Kirby C et al, Mol. Microbiol, 43: 173-86, 2002), Tyl (Devine & Boeke, Nucleic Acids Res., 22: 3765-72, 1994 and International Publication WO 95/23875), Transposon Tn7 (Craig, N L, Science. 271: 1512, 1996; Craig, N L, Review in: Curr Top Microbiol Immunol, 204:27-48, 1996), Tn/O and IS 10 (Kleckner N, et al, Curr Top Microbiol Immunol, 204:49-82, 1996), Mariner transposase (Lampe D J, et al, EMBO J., 15: 5470-9, 1996), Tel (Plasterk R H, Curr. Topics Microbiol. Immunol, 204: 125-43, 1996), P Element (Gloor, G B, Methods Mol. Biol, 260: 97-1 14, 2004), Tn3 (Ichikawa & Ohtsubo, J Biol. Chem. 265: 18829-32, 1990), bacterial insertion sequences (Ohtsubo & Sekine, Curr. Top. Microbiol. Immunol. 204: 1-26, 1996), retroviruses (Brown, et al, Proc Natl Acad Sci USA, 86:2525-9, 1989), and retrotransposon of yeast (Boeke & Corces, Annu Rev Microbiol. 43:403-34, 1989). More examples include IS5, TnlO, Tn903, IS91 1, and engineered versions of transposase family enzymes (Zhang et al, (2009) PLoS Genet. 5:e1000689. Epub 2009 Oct. 16; Wilson C. et al (2007) J. Microbiol. Methods 71:332-5) and those described in U.S. Pat. Nos. 5,925,545; 5,965,443; 6,437,109; 6,159,736; 6,406,896; 7,083,980; 7,316,903; 7,608,434; 6,294,385; 7,067,644, 7,527,966; and International Patent Publication No. WO2012103545, all of which are specifically incorporated herein by reference in their entirety. In some embodiments, the transposase is a Tn5 transposase or a hyperactive mutant thereof. In some embodiments, the transposase is a Mu transposon.

The disclosed methods can be used for tagmentation of ChIP DNA. Thus, in some examples, chromatin DNA is provided. In some examples, the chromatin DNA is cross-linking to hold any chromatin-associated factor in complex with chromatin DNA during immuneprecipitation. In some embodiments, the sample to be analyzed is contacted with a protein-nucleic acid cross-linking agent, a nucleic acid-nucleic acid cross-linking agent, a protein-protein cross-linking agent or any combination thereof. By this method, proteins and/or nucleic acids that interact with chromatin DNA become cross-linked to the chromatin DNA, such that isolation of the cross-linked proteins and/or nucleic acids also isolated as a complex with tagmented chromatin DNA to which they are bound. By this method, primary, secondary and tertiary interactions between chromatin associated factors and chromatin DNA can be discerned. In some examples, a cross-linker is a reversible cross-linker, such that the cross-linked molecules can be easily separated. In some examples, a cross-linker is a non-reversible cross-linker, such that the cross-linked molecules cannot be easily separated. In some examples, a cross-linker is light, such as UV light. In some examples, a cross linker is light activated. These cross-linkers include formaldehyde, disuccinimidyl glutarate, UV-254, psoralens and their derivatives such as aminomethyltrioxsalen, glutaraldehyde, ethylene glycol bis[succinimidylsuccinate], and other compounds known to those skilled in the art, including those described in the Thermo Scientific Pierce Cross-linking Technical Handbook, Thermo Scientific (2009) as available on the world wide web at piercenet.com/files/1601673_Cross-link_HB_Intl.pdf. In some embodiments, a chromatin-associated factor is cross-linked with chromatin DNA. In some embodiments, the chromatin-associated factor cross-linked to the chromatin DNA is contacted with a specific binding agent example after tagmentation, (for example an antibody) which may be attached to a solid support, that specifically binds to the chromatin-associated factor, for example to isolate the chromatic DNA by virtue of its interaction with the chromatin associated factor. In some embodiments, the chromatin DNA is released from the chromatin-associated factor, for example after tagmentation, and the DNA fragments produced are analyzed. In some examples, size is used to isolate the DNA fragments. Isolation of the nucleic acid fragments can be accomplished by means of an affinity molecule after the release of the fragments. For example, the material is suitable for the detection of binding sites or regions on the chromatin of low abundance chromatin-associated factors using methods such as ChIP-Seq.

In certain embodiments, the tagmented DNA fragments are purified by immobilizing the fragments on a substrate, such as a bead, membrane, or surface (e.g. a well or tube) that is coated with an affinity molecule suitable for immobilizing the nucleic acid fragments. In certain embodiments, the affinity molecule is silica or carboxyl-coated magnetic beads (SPRI beads). In certain embodiments, the library (e.g., for next generation sequencing applications, such as Illumina® sequencing (Illumina® Inc., San Diego, Calif.)) is constructed on magnetic particles. The same DNA absorbing magnetic beads can then be used to purify the resulting library. In some embodiments, a further advantage of providing an affinity surface in a well or as a bead, e.g., magnetic beads, is that the ChIP tagmentation protocol may be adapted for parallel processing of multiple samples, such as in a 96-well format or microfluidic platform, from starting chromatin material to the end of a sequencing library construction and purification. In certain embodiments, the tagmented DNA fragments are purified after they have been released from the specific chromatin-associated factor and/or antibody with which or to which the nucleic acid fragments were bound.

In some embodiments, the identity of a tagmented DNA fragment is determined by DNA sequencing, such as massively parallel sequencing. Some technologies may use cluster amplification of adapter-ligated ChIP DNA (or iChIP DNA) fragments on a solid flow cell substrate. The resulting high density array of template clusters on the flow cell surface may then be submitted to sequencing-by-synthesis in parallel using for example fluorescently labeled reversible terminator nucleotides.

Templates can be sequenced base-by-base during each read. In certain embodiments, the resulting data may be analyzed using data collection and analysis software that aligns sample sequences to a known genomic sequence. Sensitivity of this technology may depend on factors such as the depth of the sequencing run (e.g., the number of mapped sequence tags), the size of the genome, and the distribution of the target factor. By integrating a large number of short reads, highly precise binding site localization may be obtained. In certain embodiments, ChIP-Seq data can be used to locate the binding site within few tens of base pairs of the actual protein binding site, and tag densities at the binding sites may allow quantification and comparison of binding affinities of a protein to different DNA sites.

Generally, the sequencing can be performed using automated Sanger sequencing (AB13730x1 genome analyzer), pyrosequencing on a solid support (454 sequencing, Roche), sequencing-by-synthesis with reversible terminations (ILLUMINA® Genome Analyzer), sequencing-by-ligation (ABI SOLiD®) or sequencing-by-synthesis with virtual terminators (HELISCOPE®). In some embodiment the isolated tagmented fragments are analyzed, for example by determining the nucleotide sequence. In some examples, the nucleotide sequence is determined using sequencing or hybridization techniques with or without amplification.

DNA binding proteins and chromatin modifiers can be difficult to detect reliably using existing ChIP protocols because of their relative low abundance on the chromatin relative to, for example, many histone tail modifications, such as H3K4me3. ChIP performed on such abundant modifications can be very efficient and robust. A high percentage of the chromatin may be in association with modified histones. Moreover, as the DNA is wrapped tightly around histones (e.g., the nucleosome octamer), the DNA yield enriched in such studies can be relatively high, and suffices for any downstream processes.

DNA binding proteins and chromatin modifiers (or other proteins that do not bind the DNA itself, and are only a part of a complex that binds the DNA, e.g. chromatin-associated factors) are orders of magnitude less abundant across the genome and the DNA interactions of the DNA-binding proteins and associated factors are much weaker when compared to histones. The low abundance and the weak interactions with DNA are among the factors that may make a ChIP for DNA-binding proteins more susceptible to small variations and a higher sensitivity is required to obtain accurate data. Current methods with their inherent shortcomings in reproducibility and/or sensitivity may not allow for a large scale screen of DNA binding proteins and chromatin modifiers. Further factors that influence the sensitivity of the ChIP assay are, for example (1) the shearing process, which may be more sensitive to small differences when fragmenting chromatin with DNA binding proteins and may contribute to the difficulty of obtaining sufficient amounts of DNA that were in association with the DNA binding proteins; and (2) the very low amounts of DNA that can be obtained by ChIP of DNA binding proteins and chromatin modifiers may lower the overall yield. Very low yields can make it difficult to purify the DNA, a step which is often necessary for subsequent analysis. The low DNA yield generally obtained for ChIP assays involving DNA binding proteins and chromatin modifiers that are carried out using existing ChIP protocols can result in low reproducibility between repeats and can make it difficult to obtain reliable and unbiased data. ChIP assays using antibodies directed to histone modifications usually yield sufficient DNA and the yield may be, for example, about two orders of magnitude higher than the yield from ChIP assays involving DNA binding proteins and chromatin modifiers. Due to the relatively higher DNA yield, ChIP assays involving histone modifications exhibit relatively lower susceptibility to small experimental variations, which makes such assays less prone to experimental biases. Further, existing protocols can be inefficient, time consuming and difficult if not impossible to scale it up to allow parallel processing of larger sample sizes, such as is needed in high throughput screening.

Currently available ChIP protocols and/or commercially available ChIP kits are not optimal for high throughput ChIP screening. They do not provide sufficient sensitivity and/or reproducibility needed to screen large numbers of DNA binding proteins and chromatin modifiers. Provided herein, in some embodiments, are iChIP methods to obtain high quality ChIP-DNA (iChIP-DNA). In certain embodiments, the methods can be carried out easily and data can be obtained reproducibly. In certain embodiments, these methods are used to screen large numbers of DNA binding proteins and/or chromatin modifiers. In certain embodiments, the methods provided are used to screen 5, 10, 50, 100, 200, 500, 750, or 1000, or more DNA binding proteins and/or chromatin regulators (CRs) and modified forms thereof. Modified forms include, but are not limited to, mutants and post-translationally modified DNA binding proteins and/or chromatin modifiers.

In certain embodiments, the methods provided are used to screen one or more of the following DNA binding proteins and/or chromatin modifiers and modified forms thereof: AAF, ab1, ADA2, ADA-NF1, AF-1, AFP1, AhR, AIIN3, ALL-1, alpha-CBF, alpha-CP1, alpha-CP2a, alpha-CP2b, alphaHo, alphaH2-alphaH3, Alx-4, aMEF-2, AML1, AML1a, AML1b, AML1c, AML1DeltaN, AML2, AML3, AML3a, AML3b, AMY-1L, A-Myb, ANF, AP-1, AP-2alphaA, AP-2alphaB, AP-2beta, AP-2gamma, AP-3 (1), AP-3 (2), AP-4, AP-5, APC, AR, AREB6, Arnt, Arnt (774 M form), ARP-1, ATBF1-A, ATBF1-B, ATF, ATF-1, ATF-2, ATF-3, ATF-3deltaZIP, ATF-a, ATF-adelta, ATPF1, Barhl1, Barhl2, Barx1, Barx2, Bcl-3, BCL-6, BD73, beta-catenin, Bin1, B-Myb, BP1, BP2, brahma, BRCA1, Brn-3a, Brn-3b, Brn-4, BTEB, BTEB2, B-TFIID, C/EBPalpha, C/EBPbeta, C/EBPdelta, CACCbinding factor, Cart-1, CBF (4), CBF (5), CBP, CCAAT-binding factor, CCMT-binding factor, CCF, CCG1, CCK-1a, CCK-1b, CD28RC, cdk2, cdk9, Cdx-1, CDX2, Cdx-4, CFF, Chx10, CLIM1, CLIM2, CNBP, CoS, COUP, CP1, CP1A, CP1C, CP2, CPBP, CPE binding protein, CREB, CREB-2, CRE-BP1, CRE-BPa, CREMalpha, CRF, Crx, CSBP-1, CTCF, CTF, CTF-1, CTF-2, CTF-3, CTF-5, CTF-7, CUP, CUTL1, Cx, cyclin A, cyclin T1, cyclin T2, cyclin T2a, cyclin T2b, DAP, DAX1, DB1, DBF4, DBP, DbpA, DbpAv, DbpB, DDB, DDB-1, DDB-2, DEF, deltaCREB, deltaMax, DF-1, DF-2, DF-3, Dlx-1, Dlx-2, Dlx-3, DIx4 (long isoform), Dlx-4 (short isoform, Dlx-5, Dlx-6, DP-1, DP-2, DSIF, DSIF-p14, DSIF-p160, DTF, DUX1, DUX2, DUX3, DUX4, E, E12, E2F, E2F+E4, E2F+p107, E2F-1, E2F-2, E2F-3, E2F-4, E2F-5, E2F-6, E47, E4BP4, E4F, E4F1, E4TF2, EAR2, EBP-80, EC2, EF1, EF-C, EGR1, EGR2, EGR3, EIIaE-A, EIIaE-B, EIIaE-Calpha, EIIaE-Cbeta, EivF, EIf-1, EIk-1, Emx-1, Emx-2, Emx-2, En-1, En-2, ENH-bind. prot., ENKTF-1, EPAS1, epsilonF1, ER, Erg-1, Erg-2, ERR1, ERR2, ETF, Ets-1, Ets-1 deltaVil, Ets-2, Evx-1, F2F, factor 2, Factor name, FBP, f-EBP, FKBP59, FKHL18, FKHRL1P2, Fli-1, Fos, FOXB1, FOXC1, FOXC2, FOXD1, FOXD2, FOXD3, FOXD4, FOXE1, FOXE3, FOXF1, FOXF2, FOXG1a, FOXG1b, FOXG1c, FOXH1, FOXI1, FOXJ1a, FOXJ1b, FOXJ2 (long isoform), FOXJ2 (short isoform), FOXJ3, FOXK1a, FOXK1b, FOXK1c, FOXL1, FOXM1a, FOXM1b, FOXM1c, FOXN1, FOXN2, FOXN3, FOX01a, FOX01b, FOXO2, FOXO3a, FOXO3b, FOXO4, FOXP1, FOXP3, Fra-1, Fra-2, FTF, FTS, G factor, G6 factor, GABP, GABP-alpha, GABP-beta1, GABP-beta2, GADD 153, GAF, gammaCMT, gammaCAC1, gammaCAC2, GATA-1, GATA-2, GATA-3, GATA-4, GATA-5, GATA-6, Gbx-1, Gbx-2, GCF, GCMa, GCNS, GF1, GLI, GLI3, GR alpha, GR beta, GRF-1, Gsc, Gscl, GT-IC, GT-IIA, GT-IIBalpha, GT-IIBbeta, H1TF1, H1TF2, H2RIIBP, H4TF-1, H4TF-2, HAND1, HAND2, HB9, HDAC1, HDAC2, HDAC3, hDaxx, heat-induced factor, HEB, HEB1-p67, HEB1-p94, HEF-1 B, HEF-1T, HEF-4C, HENT, HEN2, Hesx1, Hex, HIF-1, HIF-1alpha, HIF-1beta, HiNF-A, HiNF-B, HINF-C, HINF-D, HiNF-D3, HiNF-E, HiNF-P, HIP1, HIV-EP2, Hlf, HLTF, HLTF (Met123), HLX, HMBP, HMG I, HMG I(Y), HMG Y, HMGI-C, HNF-1A, HNF-1B, HNF-1C, HNF-3, HNF-3alpha, HNF-3beta, HNF-3gamma, HNF4, HNF-4alpha, HNF4alpha1, HNF-4alpha2, HNF-4alpha3, HNF-4alpha4, HNF4gamma, HNF-6alpha, hnRNP K, HOX11, HOXA1, HOXA10, HOXA10 PL2, HOXA11, HOXA13, HOXA2, HOXA3, HOXA4, HOXA5, HOXA6, HOXA7, HOXA9A, HOXA9B, HOXB-1, HOXB13, HOXB2, HOXB3, HOXB4, HOXBS, HOXB6, HOXA5, HOXB7, HOXB8, HOXB9, HOXC10, HOXC11, HOXC12, HOXC13, HOXC4, HOXC5, HOXC6, HOXC8, HOXC9, HOXD10, HOXD11, HOXD12, HOXD13, HOXD3, HOXD4, HOXD8, HOXD9, Hp55, Hp65, HPX42B, HrpF, HSF, HSF1 (long), HSF1 (short), HSF2, hsp56, Hsp90, IBP-1, ICER-II, ICER-ligamma, ICSBP, Id1, Id1 H′, Id2, Id3, Id3/Heir-1, IF1, IgPE-1, IgPE-2, IgPE-3, IkappaB, IkappaB-alpha, IkappaB-beta, IkappaBR, II-1 RF, IL-6 RE-BP, 11-6 RF, INSAF, IPF1, IRF-1, IRF-2, ir1B, IRX2a, Irx-3, Irx-4, ISGF-1, ISGF-3, ISGF3alpha, ISGF-3gamma, 1st-1, ITF, ITF-1, ITF-2, JRF, Jun, JunB, JunD, kappay factor, KBP-1, KER1, KER-1, Kox1, KRF-1, Ku autoantigen, KUP, LBP-1, LBP-1a, LBX1, LCR-F1, LEF-1, LEF-1B, LF-A1, LHX1, LHX2, LHX3a, LHX3b, LHXS, LHX6.1a, LHX6.1b, LIT-1, Lmo1, Lmo2, LMX1A, LMX1B, L-My1 (long form), L-My1 (short form), L-My2, LSF, LXRalpha, LyF-1, LyI-1, M factor, Mad1, MASH-1, Max1, Max2, MAZ, MAZ1, MB67, MBF1, MBF2, MBF3, MBP-1 (1), MBP-1 (2), MBP-2, MDBP, MEF-2, MEF-2B, MEF-2C (433 AA form), MEF-2C (465 AA form), MEF-2C (473 M form), MEF-2C/delta32 (441 AA form), MEF-2D00, MEF-2DOB, MEF-2DA0, MEF-2DA′0, MEF-2DAB, MEF-2DA′B, Meis-1, Meis-2a, Meis-2b, Meis-2c, Meis-2d, Meis-2e, Meis3, Meox1, Meox1a, Meox2, MHox (K-2), Mi, MIF-1, Miz-1, MM-1, MOP3, MR, Msx-1, Msx-2, MTB-Zf, MTF-1, mtTF1, Mxi1, Myb, Myc, Myc 1, Myf-3, Myf-4, Myf-5, Myf-6, MyoD, MZF-1, NC1, NC2, NCX, NELF, NER1, Net, NF III-a, NF NF NF-1, NF-1A, NF-1B, NF-1X, NF-4FA, NF-4FB, NF-4FC, NF-A, NF-AB, NFAT-1, NF-AT3, NF-Atc, NF-Atp, NF-Atx, NfbetaA, NF-CLE0a, NF-CLE0b, NFdeltaE3A, NFdeltaE3B, NFdeltaE3C, NFdeltaE4A, NFdeltaE4B, NFdeltaE4C, Nfe, NF-E, NF-E2, NF-E2 p45, NF-E3, NFE-6, NF-Gma, NF-GMb, NF-IL-2A, NF-IL-2B, NF-jun, NF-kappaB, NF-kappaB(-like), NF-kappaB1, NF-kappaB1, precursor, NF-kappaB2, NF-kappaB2 (p49), NF-kappaB2 precursor, NF-kappaE1, NF-kappaE2, NF-kappaE3, NF-MHCIIA, NF-MHCIIB, NF-muE1, NF-muE2, NF-muE3, NF-S, NF-X, NF-X1, NF-X2, NF-X3, NF-Xc, NF-YA, NF-Zc, NF-Zz, NHP-1, NHP-2, NHP3, NHP4, NKX2-5, NKX2B, NKX2C, NKX2G, NKX3A, NKX3A v1, NKX3A v2, NKX3A v3, NKX3A v4, NKX3B, NKX6A, Nmi, N-Myc, N-Oct-2alpha, N-Oct-2beta, N-Oct-3, N-Oct-4, N-Oct-5a, N-Oct-5b, NP-TCII, NR2E3, NR4A2, Nrf1, Nrf-1, Nrf2, NRF-2beta1, NRF-2gamma1, NRL, NRSF form 1, NRSF form 2, NTF, 02, OCA-B, Oct-1, Oct-2, Oct-2.1, Oct-2B, Oct-2C, Oct-4A, Oct4B, Oct-5, Oct-6, Octa-factor, octamer-binding factor, oct-B2, oct-B3, Otx1, Otx2, OZF, p107, p130, p28 modulator, p300, p38erg, p45, p49erg,-p53, p55, p55erg, p65delta, p67, Pax-1, Pax-2, Pax-3, Pax-3A, Pax-3B, Pax-4, Pax-5, Pax-6, Pax-6/Pd-5a, Pax-7, Pax-8, Pax-8a, Pax-8b, Pax-8c, Pax-8d, Pax-8e, Pax-8f, Pax-9, Pbx-1a, Pbx-1b, Pbx-2, Pbx-3a, Pbx-3b, PC2, PC4, PC5, PEA3, PEBP2alpha, PEBP2beta, Pit-1, PITX1, PITX2, PITX3, PKNOX1, PLZF, PO-B, Pontin52, PPARalpha, PPARbeta, PPARgamma1, PPARgamma2, PPUR, PR, PR A, pRb, PRD1-BF1, PRD1-BFc, Prop-1, PSE1, P-TEFb, PTF, PTFalpha, PTFbeta, PTFdelta, PTFgamma, Pu box binding factor, Pu box binding factor (BJA-B), PU.1, PuF, Pur factor, R1, R2, RAR-alpha1, RAR-beta, RAR-beta2, RAR-gamma, RAR-gamma1, RBP60, RBP-Jkappa, Rel, RelA, RelB, RFX, RFX1, RFX2, RFX3, RFXS, RF-Y, RORalpha1, RORalpha2, RORalpha3, RORbeta, RORgamma, Rox, RPF1, RPGalpha, RREB-1, RSRFC4, RSRFC9, RVF, RXR-alpha, RXR-beta, SAP-1a, SAP1b, SF-1, SHOX2a, SHOX2b, SHOXa, SHOXb, SHP, SIII-p110, SIII-p15, SIII-p18, SIM′, Six-1, Six-2, Six-3, Six-4, Six-5, Six-6, SMAD-1, SMAD-2, SMAD-3, SMAD-4, SMAD-5, SOX-11, SOX-12, Sox-4, Sox-5, SOX-9, Sp1, Sp2, Sp3, Sp4, Sph factor, Spi-B, SPIN, SRCAP, SREBP-1a, SREBP-1b, SREBP-1c, SREBP-2, SRE-ZBP, SRF, SRY, SRP1, Staf-50, STAT1alpha, STAT1beta, STAT2, STAT3, STAT4, STATE, T3R, T3R-alpha1, T3R-alpha2, T3R-beta, TAF(I)110, TAF(I)48, TAF(I)63, TAF(II)100, TAF(II)125, TAF(II)135, TAF(II)170, TAF(II)18, TAF(II)20, TAF(II)250, TAF(II)250Delta, TAF(II)28, TAF(II)30, TAF(II)31, TAF(II)55, TAF(II)70-alpha, TAF(II)70-beta, TAF(II)70-gamma, TAF-I, TAF-II, TAF-L, Tal-1, Tal-1beta, Tal-2, TAR factor, TBP, TBX1A, TBX1B, TBX2, TBX4, TBXS (long isoform), TBXS (short isoform), TCF, TCF-1, TCF-1A, TCF-1B, TCF-1C, TCF-1D, TCF-1E, TCF-1F, TCF-1G, TCF-2alpha, TCF-3, TCF-4, TCF-4(K), TCF-4B, TCF-4E, TCFbeta1, TEF-1, TEF-2, tel, TFE3, TFEB, TFIIA, TFIIA-alpha/beta precursor, TFIIA-alpha/beta precursor, TFIIA-gamma, TFIIB, TFIID, TFIIE, TFIIE-alpha, TFIIE-beta, TFIIF, TFIIF-alpha, TFIIF-beta, TFIIH, TFIIH*, TFIIH-CAK, TFIIH-cyclin H, TFIIH-ERCC2/CAK, TFIIH-MATT, TFIIH-MO15, TFIIH-p34, TFIIH-p44, TFIIH-p62, TFIIH-p80, TFIIH-p90, TFII-I, Tf-LF1, Tf-LF2, TGIF, TGIF2, TGT3, THRA1, TIF2, TLE1, TLX3, TMF, TR2, TR2-11, TR2-9, TR3, TR4, TRAP, TREB-1, TREB-2, TREB-3, TREF1, TREF2, TRF (2), TTF-1, TXRE BP, TxREF, UBF, UBP-1, UEF-1, UEF-2, UEF-3, UEF-4, USF1, USF2, USF2b, Vav, Vax-2, VDR, vHNF-1A, vHNF-1B, vHNF-1C, VITF, WSTF, WT1, WT1I, WT1 I-KTS, WT1 I-del2, WT1-KTS, WT1-del2, X2BP, XBP-1, XW-V, XX, YAF2, YB-1, YEBP, YY1, ZEB, ZF1, ZF2, ZFX, ZHX1, ZIC2, ZID, ZNF174. ASH1L, ASH2, ATF2, ASXL1, BAP1, bcllO, Bmil, BRG1, CARM1, KAT3A/CBP, CDC73, CHD1, CHD2, CTCF, DNMT1, DOTL1, EHMT1, ESET, EZH1, EZH2, FBXL10, FRP(Plu-1), HDAC1, HDAC2, HMGA1, hnRNPA1, HP1 gamma, Hset1b, Jarid1A, Jarid1C, KIAA1718_JHDM1D, KAT5, KMT4, LSD1, NFKB P100, NSD2, MBD2, MBD3, MLL2, MLL4, P300, pRB, RbAP46/48, RBP1, RbBP5, RING1B, RNApolII P S2, RNApolII P S5, ROC1, sap30, setDB 1, Sf3b1, SIRT1, Sirt6, SMYD1, SP1, SUV39H1, SUZ12, TCF4, TET1, TRRAP, TRX2, WDR5, WDR77, and/or YY1. Antibodies for these DNA binding proteins and/or chromatin modifiers are commercially available.

Low abundance chromatin-associated factors, as used herein, are factors that can be found at one or more sites on the chromatin and/or that may associate with chromatin in a transient manner. Examples of low abundance chromatin-associated factors include, but are not limited to, transcription factors (e.g., tumor suppressors, oncogenes, cell cycle regulators, development and/or differentiation factors, general transcription factors (TFs)), activator (e.g., histone acetyl transferase (HAT)) complexes, repressor (e.g., histone deacetylase (HDAC)) complexes, co-activators, co-repressors, other chromatin-remodelers, e.g., histone (de-) methylases, DNA methylases, replication factors and the like. Such factors may interact with the chromatin (DNA, histones) at particular phases of the cell cycle (e.g., G1, S, G2, M-phase), upon certain environmental cues (e.g., growth and other stimulating signals, DNA damage signals, cell death signals) upon transfection and transient or stable expression (e.g., recombinant factors) or upon infection (e.g., viral factors). Abundant factors are constituents of the chromatin, e.g., histones. Histones may be modified at histone tails through posttranslational modifications which alter their interaction with DNA and nuclear proteins and influence for example gene regulation, DNA repair and chromosome condensation. The H3 and H4 histones have long tails protruding from the nucleosome which can be covalently modified, for example by methylation, acetylation, phosphorylation, ubiquitination, sumoylation, citrullination and ADP-ribosylation. The core of the histones H2A and H2B can also be modified. Combinations of modifications are thought to constitute the so-called “histone code” (Strahl and Allis (2000) Nature 403 (6765): 41-5; Jenuwein and Allis (2001) Science 293 (5532): 1074-80).

In certain embodiments, the disclosed methods are provided that allow sample processing in a high-throughput manner. For example, 10, 50, 100, 200, 500, 750, 1000, or more chromatin-associated factors and/or chromatin modifications may be immuno-precipitated and/or analyzed in parallel. In one embodiment, up to 96 samples may be processed at once, using e.g., a 96-well plate. In other embodiments, fewer or more samples may be processed, using e.g., 6-well, 12-well, 32-well, 384-well or 1536-well plates. In some embodiments, ChIP methods are provided that can be carried out in tubes, such as, for example, common 1.5 ml, 2.0 ml, 15 ml, 50 ml size tubes. These tubes may be arrayed in tube racks, floats or other holding devices.

For any one of the embodiments described herein, the immune-precipitated chromatin may be prepared from harvested cells (e.g., subsequently subjected to sonication). In certain embodiments, the immune-precipitated chromatin may be prepared from a single sample of about 1 million to about 20 million cells, or more. In certain embodiments, immune-precipitated chromatin may be prepared from a single sample of about 1 cell to about 1 million cells. In particular embodiments, a sample may comprise about 1 cell, about 2 cells, about 3 cells, about 5 cells, about 10 cells, about 25, about 50 cells, about 100 cells, about 150 cells, about 200 cells, about 300 cells, about 400 cells, about 500 cells, about 1000 cells, about 2000 cells, about 3000 cells, about 4000 cells, about 5000 cells, about 10,000 cells, about 20,000 cells, about 30,000 cells, about 40,000 cells, about 50,000 cells, about 100,000 cells, about 200,000 cells, about 300,000 cells, about 400,000 cells, about 500,000 cells, or about 1,000,000 cells. In some embodiments, a sample may comprise about 1 cell to about 10,000 cells, or about 10,000 cells to about 100,000 cells, or more. In some embodiments, immobilization of the factor-bound sheared chromatin fragments and subsequent eluted complex-free nucleic acid fragments using affinity-based immobilization methods described herein (e.g., using beads or coated surfaces of reaction containers) allows robotic dispensing and aspiration of wash solutions and elution buffers, as well as sample transfer into new reaction containers (e.g., multi-well/micro plates).

Specific DNA sites that are in direct physical interaction with transcription factors and other proteins, such as histones, may be isolated by, which produces a library of target DNA sites bound by a protein in vivo. In some embodiments, massively parallel sequence analyses may be used in conjunction with whole-genome sequence databases to analyze the interaction pattern of a protein of interest (e.g., transcription factors, polymerases or transcriptional machinery) with DNA or to analyze the pattern of an epigenetic chromatin modification of interest (e.g., histone modifications or DNA modifications).

ChIP may be used, in some embodiments, to selectively enrich for DNA sequences bound by a particular protein in living cells by cross-linking DNA-protein complexes and using an antibody that is specific against a protein of interest. After precipitation of chromatin, oligonucleotide adapters may be added to the small stretches of DNA that are bound to the protein of interest to enable massively parallel sequencing. After size selection, the resulting DNA fragments can be sequenced simultaneously using, for example, a genome sequencer. A single sequencing run can scan for genome-wide associations with high resolution.

In certain methods, analysis of chromatin is biased to the open regions of chromatin (see for example Buenrostro et al. Nature Methods 10, pp 1213-1218 (2013)). To overcome such bias toward open chromatin, the inventors have developed several techniques. Thus in certain aspect embodiments, disclosed are methods of shearing and tagging chromatin bound DNA without significant bias to open chromatin. In such methods, prior to contact with the one or more transposomes, the chromatin bound DNA is loosened, for example to make closed chromatin accessible. In some examples, the DNA is loosened by pre-nicking the chromatin with an MNase to induce single or double strand breaks in the DNA. In some examples, the DNA is loosened by contacting the chromatin DNA with a restriction enzyme whose recognition sites are locate with high concentration in closed chromatin (e.g. an AT rich 6 cutter). In some examples, the chromatin is minimally sheared, to just loosen the chromatin (e.g. on a Covaris® system). In some examples, the chromatin is loosened using a change in buffer conditions, for example high salt conditions.

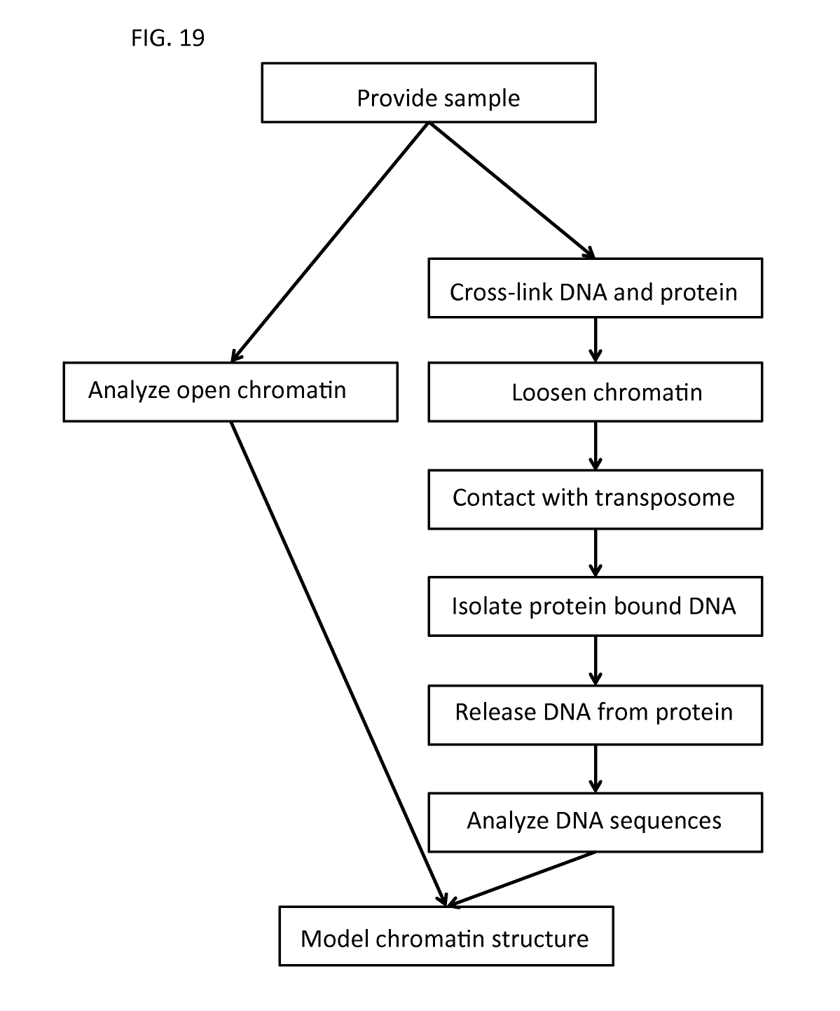

In some embodiments, the methods disclosed herein are used to simultaneously measure the open chromatin and the proteins bound to chromatin, including open and closed chromatin. FIG. 19 is a flow chart showing in example of such an analysis. In such methods, a sample of chromatin DNA, such as chromatin DNA crosslinked to proteins is provided. The sample can be divided and a portion analyzed according to the methods disclosed herein, including non-biased tagging and shearing, while another portion of the sample is analyzed to determine the DNA-binding proteins and nucleosome position in open chromatin (for example using the methods provided in Buenrostro et al. Nature Methods 10, pp 1213-1218 (2013)). The combination of the two methods can be used to model chromatin structure.

The disclosed methods are also particularly suited to monitoring disease states, such as disease state in an organism, for example a plant or an animal subject, such as a mammalian subject, for example a human subject. Certain disease states may be caused and/or characterized differential binding or proteins and/or nucleic acids to chromatin DNA in vivo. For example, certain interactions may occur in a diseased cell but not in a normal cell. In other examples, certain interactions may occur in a normal cell but not in diseased cell. Thus, using the disclosed methods a profile of the interaction between a in vivo, can be correlated with a disease state.

Accordingly, aspects of the disclosed methods relate to correlating the interactions of a target nucleic acid with proteins and/or nucleic acid with a disease state, for example cancer, or an infection, such as a viral or bacterial infection. It is understood that a correlation to a disease state could be made for any organism, including without limitation plants, and animals, such as humans.

The interaction profile correlated with a disease can be used as a “fingerprint” to identify and/or diagnose a disease in a cell, by virtue of having a similar “fingerprint.” The profile of chromatin associated factors and chromatin DNA can be used to identify binding proteins and/or nucleic acids that are relevant in a disease state such as cancer, for example to identify particular proteins and/or nucleic acids as potential diagnostic and/or therapeutic targets. In addition, the profile can be used to monitor a disease state, for example to monitor the response to a therapy, disease progression and/or make treatment decisions for subjects.

The ability to obtain an interaction profile allows for the diagnosis of a disease state, for example by comparison of the profile present in a sample with the correlated with a specific disease state, wherein a similarity in profile indicates a particular disease state.

Accordingly, aspects of the disclosed methods relate to diagnosing a disease state based on interaction profile correlated with a disease state, for example cancer, or an infection, such as a viral or bacterial infection. It is understood that a diagnosis of a disease state could be made for any organism, including without limitation plants, and animals, such as humans.

Aspects of the present disclosure relate to the correlation of an environmental stress or state with an interaction profile, for example a whole organism, or a sample, such as a sample of cells, for example a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value.

In some embodiments, the disclosed methods can be used to screen chemical libraries for agents that modulate interaction profiles, for example that alter the interaction profile from an abnormal one, for example correlated to a disease state to one indicative of a disease free state. By exposing cells, or fractions thereof (such as nuclear extract), tissues, or even whole animals, to different members of the chemical libraries, and performing the methods described herein, different members of a chemical library can be screened for their effect on interaction profiles simultaneously in a relatively short amount of time, for example using a high throughput method.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

Appropriate agents can be contained in libraries, for example, synthetic or natural compounds in a combinatorial library. Numerous libraries are commercially available or can be readily produced; means for random and directed synthesis of a wide variety of organic compounds and biomolecules, including expression of randomized oligonucleotides, such as antisense oligonucleotides and oligopeptides, also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds.

Preparation and screening of combinatorial libraries is well known to those of skill in the art. Libraries (such as combinatorial chemical libraries) useful in the disclosed methods include, but are not limited to, peptide libraries (see, e.g., U.S. Pat. No. 5,010,175; Furka, Int. J. Pept. Prot. Res., 37:487-493, 1991; Houghton et al, Nature, 354:84-88, 1991; PCT Publication No. WO 91/19735), (see, e.g., Lam et al., Nature, 354:82-84, 1991; Houghtenet al., Nature, 354:84-86, 1991), and combinatorial chemistry-derived molecular library made of D- and/or L-configuration amino acids, phosphopeptides (including, but not limited to, members of random or partially degenerate, directed phosphopeptide libraries; see, e.g., Songyang et al., Cell, 72:767-778, 1993), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), small organic or inorganic molecules (such as, so-called natural products or members of chemical combinatorial libraries), molecular complexes (such as protein complexes), or nucleic acids, encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Natl Acad. Sa. USA, 90:6909-6913, 1993), vinylogous polypeptides (Hagihara et al., J. Am. Chem. Soc, 114:6568, 1992), nonpeptidal peptidomimetics with glucose scaffolding (Hirschmann et al., J. Am. Chem. Soc, 114:9217-9218, 1992), analogous organic syntheses of small compound libraries (Chen et al., J. Am. Chem. Soc, 116:2661, 1994), oligo carbamates (Cho et al., Science, 261:1303, 1003), and/or peptidyl phosphonates (Campbell et al., J. Org. Chem., 59:658, 1994), nucleic acid libraries (see Sambrook et al. Molecular Cloning, A Laboratory Manual, Cold Springs Harbor Press, N Y., 1989; Ausubel et al., Current Protocols m Molecular Biology, Green Publishing Associates and Wiley Interscience, N. Y., 1989), peptide nucleic acid libraries (see, e.g., U.S. Pat. No. 5,539,083), antibody libraries (see, e.g., Vaughn et al., Nat. Biotechnol, 14:309-314, 1996; PCT App. No. PCT/US96/10287), carbohydrate libraries (see, e.g., Liang et al., Science, 274:1520-1522, 1996; U.S. Pat. No. 5,593,853), small organic molecule libraries (see, e.g., benzodiazepines, Baum, C&EN, January 18, page 33, 1993; isoprenoids, U.S. Pat. No. 5,569,588; thiazolidionones and methathiazones, U.S. Pat. No. 5,549,974; pyrrolidines, U.S. Pat. Nos. 5,525,735 and 5,519,134; morpholino compounds, U.S. U.S. Pat. No. 5,506,337; benzodiazepines, U.S. Pat. No. 5,288,514) and the like.

Libraries useful for the disclosed screening methods can be produced in a variety of manners including, but not limited to, spatially arrayed multipin peptide synthesis (Geysen, et al., Proc. Natl. Acad. Sa., 81(13):3998-4002, 1984), “tea bag” peptide synthesis (Houghten, Proc. Natl. Acad. Sa., 82(15):5131-5135, 1985), phage display (Scott and Smith, Science, 249:386-390, 1990), spot or disc synthesis (Dittrich et al., Bworg. Med. Chem. Lett., 8(17):2351-2356, 1998), or split and mix solid phase synthesis on beads (Furka et al., Int. J. Pept. Protein Res., 37(6):487-493, 1991; Lam et al., Chem. Rev., 97 (2):411-448, 1997).

Devices for the preparation of combinatorial libraries are also commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, for example, ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Libraries can include a varying number of compositions (members), such as up to about 100 members, such as up to about 1,000 members, such as up to about 5,000 members, such as up to about 10,000 members, such as up to about 100,000 members, such as up to about 500,000 members, or even more than 500,000 members. In one example, the methods can involve providing a combinatorial chemical or peptide library containing a large number of potential therapeutic compounds. Such combinatorial libraries are then screened by the methods disclosed herein to identify those library members (particularly chemical species or subclasses) that display a desired characteristic activity.

The compounds identified using the methods disclosed herein can serve as conventional “lead compounds” or can themselves be used as potential or actual therapeutics. In some instances, pools of candidate agents can be identified and further screened to determine which individual or subpools of agents in the collective have a desired activity.

Control reactions can be performed in combination with the libraries. Such optional control reactions are appropriate and can increase the reliability of the screening. Accordingly, disclosed methods can include such a control reaction. The control reaction may be a negative control reaction that measures the transcription factor activity independent of a transcription modulator. The control reaction may also be a positive control reaction that measures transcription factor activity in view of a known transcription modulator.

Compounds identified by the disclosed methods can be used as therapeutics or lead compounds for drug development for a variety of conditions. Because gene expression is fundamental in all biological processes, including cell division, growth, replication, differentiation, repair, infection of cells, etc., the ability to monitor transcription factor activity and identify compounds which modulator their activity can be used to identify drug leads for a variety of conditions, including neoplasia, inflammation, allergic hypersensitivity, metabolic disease, genetic disease, viral infection, bacterial infection, fungal infection, or the like. In addition, compounds identified that specifically target transcription factors in undesired organisms, such as viruses, fungi, agricultural pests, or the like, can serve as fungicides, bactericides, herbicides, insecticides, and the like. Thus, the range of conditions that are related to transcription factor activity includes conditions in humans and other animals, and in plants, such as agricultural applications.

Appropriate samples for use in the methods disclosed herein include any conventional biological sample obtained from an organism or a part thereof, such as a plant, animal, bacteria, and the like. In particular embodiments, the biological sample is obtained from an animal subject, such as a human subject. A biological sample is any solid or fluid sample obtained from, excreted by or secreted by any living organism, including without limitation, single celled organisms, such as bacteria, yeast, protozoans, and amebas among others, multicellular organisms (such as plants or animals, including samples from a healthy or apparently healthy human subject or a human patient affected by a condition or disease to be diagnosed or investigated, such as cancer). For example, a biological sample can be a biological fluid obtained from, for example, blood, plasma, serum, urine, bile, ascites, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as a rheumatoid arthritis, osteoarthritis, gout or septic arthritis). A sample can also be a sample obtained from any organ or tissue (including a biopsy or autopsy specimen, such as a tumor biopsy) or can include a cell (whether a primary cell or cultured cell) or medium conditioned by any cell, tissue or organ. Exemplary samples include, without limitation, cells, cell lysates, blood smears, cytocentrifuge preparations, cytology smears, bodily fluids (e.g., blood, plasma, serum, saliva, sputum, urine, bronchoalveolar lavage, semen, etc.), tissue biopsies (e.g., tumor biopsies), fine-needle aspirates, and/or tissue sections (e.g., cryostat tissue sections and/or paraffin-embedded tissue sections). In other examples, the sample includes circulating tumor cells (which can be identified by cell surface markers). In particular examples, samples are used directly (e.g., fresh or frozen), or can be manipulated prior to use, for example, by fixation (e.g., using formalin) and/or embedding in wax (such as formalin-fixed paraffin-embedded (FFPE) tissue samples). It will appreciated that any method of obtaining tissue from a subject can be utilized, and that the selection of the method used will depend upon various factors such as the type of tissue, age of the subject, or procedures available to the practitioner. Standard techniques for acquisition of such samples are available. See, for example Schluger et al., J. Exp. Med. 176:1327-33 (1992); Bigby et al., Am. Rev. Respir. Dis. 133:515-18 (1986); Kovacs et al., NEJM 318:589-93 (1988); and Ognibene et al., Am. Rev. Respir. Dis. 129:929-32 (1984).

### C. Kits

The reagents disclosed herein can be supplied in the form of a kit for use in the tagmentation of chromatin DNA. In such a kit, an appropriate amount of one or more cross-linking agent; a first specific binding agent that binds to a chromatin-associated factor, or is coated with a molecule that binds to the first affinity molecule, to form a first affinity surface, a transposase, a transposon comprising a first DNA molecule comprising a first transposase recognition site; and a second DNA molecule comprising a second transposase recognition site are provided in one or more containers or held on a substrate. The reagents can be provided suspended in an aqueous solution or as a freeze-dried or lyophilized powder, for instance. The container(s) which are supplied can be any conventional container that is capable of holding the supplied form, for instance, microfuge tubes, ampoules, or bottles. The kits can include either labeled or unlabeled nucleic acids.

The kit can further include one or more of a buffer solutions, each in separate packaging, such as a container. Additional components in some kits include instructions for carrying out the assay. Instructions permit the tester to determine whether expression levels are elevated, reduced, or unchanged in comparison to a control sample. Reaction vessels and auxiliary reagents, such as chromogens, buffers, enzymes, etc., can also be included in the kits. The instructions can include directions for obtaining a sample, processing the sample

The following example is provided to illustrate certain particular features and/or embodiments. This example should not be construed to limit the invention to the particular features or embodiments described.

### EXAMPLE

### A Protocol for Tagging and Shearing Chromatin DNA for Chromatin Immunoprecipitation Followed by High Throughput Sequencing

### Cell Lysis

Resuspended cells in PBS
Pipette 2 ul of resuspended cells into a new 0.7 ml PCR tube
Pipette 2 ul of 2× lysis buffer
Incubate on ice for 10 min to lyse cells

### Tagmentation

Shear chromatin and insert sequencing adapters
   Make up tagmentation reaction as follows:
   add 6 ul UltraPure H2O to lysis reaction
   add 12.5 ul Nextera TD buffer
   add 2.5 Nextera TDE1 enzyme
   total volume: 25 ul
Mix thoroughly, briefly spin down
Incubate reaction at 37 C for 1 hour

### Chromatin-Immunoprecipitation (ChIP)

Prepare Protein AG Magnetic Beads
   Place required quantity on magnet, wait until solution is clear
   Aspirate supernatant
   Wash each tube with 1 mL binding buffer(?), repeat once
   Resuspend in binding buffer using 100 ul*number of antibodies to be used
Prepare antibody and AG bead mix
   add appropriate amount of Ab
   put at 4C on rocker for 1 h
   put at RT on rocker for 15 min
   put on magnet, take off supernatant
   wash in 1 ml binding buffer
   resuspend in 150 ul*desired # of IP reaction
Take 150 ul of conjugated Ab/beads and combine with tagmented sample
Seal sample well and put on rotating rocker at 4C overnight

### Clean ChIP

Centrifuge sample after overnight incubation
Place on magnet, let solution clear for 5 min
Remove supernatant
Wash 4× using 120 ul low salt RIPA buffer
Wash 2× using 120 ul high salt RIPA buffer
Wash 2× using 120 ul LiCl buffer(?)
Wash 2× using 120 ul 10 mM Tris-HCl
Elute DNA off beads using 50 ul elution buffer
Remove RNA and proteins
   Add 5 ul RNase to each sample, mix by pipetting
   Incubate at 37 C for 5 min
   Add 3 ul Protinase K to each sample
   Incubate at 37 C for 2 hours, then 65 C for 5 min

### PCR Amplification

During incubation prepare 0.7 volumes Ampure XP beads for each reaction according to manufacturer suggestion
Clean up nextera reaction by adding 0.7 volumes Ampure XP beads to select fragments >200 bp
Perform cleanup reaction according to manual for Ampure XP beads
Elute in 10 ul UltraPure H2O
Amplify tagmented DNA using PCR. In a new tube prepare the following reaction
   add 2.5 ul each index Illumina primer (N7XX and N5XX, respectively)
   add 2.5 ul Nextera NPC primer cocktail
   add 7.5 ul Nextera NPM PCR mix
   add 7.5 ul eluate from Nextera tagmentation reaction
Perform PCR using the following program
   72C for 3 minutes
   98 C for 30 seconds
   up to 20 cycles of: (can stop earlier, pause after desired # of cycles and check sample on gel)
   98 C for 10 seconds
   63 C for 30 seconds
   72 C for 3 minutes
   end/hold at 4C

### PCR Cleanup

Clean up PCR reaction product using 0.7 volumes of AmpureXP beads according to manufacturer instructions.

### Sequencing Library Preparation

Measure sample concentration after cleanup.
Dilute each to sample to 2 nM.
Combine samples in 10 ul total volume to achieve desired read coverage for each sample
   E.g.: If equal coverage is desired from two samples, use 5 ul and 5 ul.
Immediately before sequencing pooled libraries:
Denature pool with 1 volume 0.2N NaOH
Incubate 5 min at room temperature.
Dilute to 1 mL with illumina HT1 buffer
move 400 ul of dilution to a new tube and add another 600 ul of HT1 for a 8 pM sample
load 600 ul in sequencing cartridge

### 2×SC Lysis Buffer

### 100 mM Tris-HCl pH7.5

### 300 mM NaCl

### 2% Triton® X-100

0.2% sodium deoxycholate

### 10 mM CaCl2

### H2O

### Elution Buffer

### LiCl Buffer

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of this disclosure and these claims.

1. A method for shearing and tagging chromatin DNA,
comprising: contacting chromatin DNA, under conditions that permit integration of a transposon into chromatin DNA, with at least one transposome, the transposome comprising:
at least one transposase; and
a transposon comprising:
a first DNA molecule comprising a first transposase recognition site; and
a second DNA molecule comprising a second transposase recognition site wherein the at least one transposase integrates the first and second DNA molecules into chromatin DNA,
thereby shearing and tagging chromatin DNA with the first and second DNA molecules.

2. The method of claim 1, wherein the first and/or second DNA molecule further comprises a barcode, a sequencing adapter, or a universal priming site.

3. (canceled)

4. (canceled)

5. The method of claim 1, wherein the at least one transposase comprises a Tn5 transposase, a Mu transposase, an IS5 transposase, an IS91 transposase, or a combination thereof.

6. (canceled)

7. (canceled)

8. The method of claim 1, wherein the least one transposome comprises at least two different transposomes, and wherein the different transposomes integrate different DNA sequences into the chromatin DNA.

9. The method of claim 8, wherein the chromatin DNA comprises chromatin DNA from a single cell.

10. The method of claim 1, further comprising providing chromatin DNA.

11. The method of claim 10, wherein providing chromatin DNA comprises providing cross-linked chromatin, wherein the chromatin is cross-linked to chromatin associated factors.

12. The method of claim 11, further comprising cross-linking chromatin to chromatin associated factors to chromatin DNA.

13. The method of claim 12, further comprising contacting the chromatin-associated factor cross-linked to the chromatin DNA with a specific binding agent that specifically binds to the chromatin-associated factor.

14. The method of claim 13, further comprising releasing the nucleic acid from the chromatin-associated factor.

15. The method of claim 13, wherein the specific binding agent is attached to a solid support.

16. The method of claim 13, wherein the specific binding agent is an antibody.

17. The method of claim 1, further comprising isolating DNA fragments produced.

18. The method of claim 17, wherein the DNA fragments are isolated based on size.

19. The method of claim 18, further comprising analyzing the isolated nucleic acid fragments.

20. The method of claim 19, wherein analyzing the isolated nucleic acid fragments comprises determining the nucleotide sequence.

21. The method of claim 20, wherein the nucleotide sequence is determined using sequencing or hybridization techniques with or without amplification.

22. The method of claim 1, further comprising increasing the accessibility of closed chromatin to the at least one transposome.

23. The method of claim 22, wherein increasing the accessibility of closed chromatin to the at least one transposome, comprises one or more of contacting the chromatin DNA with MNase, contacting the chromatin DNA with a restriction enzyme whose recognition sites are located with high concentration in closed chromatin, minimally shearing the chromatin DNA or exposing the chromatin DNA to high salt conditions.

24. A method for preparing a library of sheared and tagged chromatin DNA fragments comprising the method of claim 1.

25. A chromatin immuno-precipitation tagementation kit, the kit comprising:
a cross-linking agent;
a first specific binding agent that binds to a chromatin-associated factor, or is coated with
a molecule that binds to the first affinity molecule, to form a first affinity surface, and a transposase; and
the transposon comprising:
a first DNA molecule comprising a first transposase recognition site; and
a second DNA molecule comprising a second transposase recognition site,
wherein the transposase integrates the first and second DNA molecules into chromatin DNA.

26. The kit of claim 25, wherein the first and/or second DNA molecule further comprises a barcode, a sequencing adaptor, or a universal priming site.

27. (canceled)

28. (canceled)

29. The kit of claim 25, wherein the transposase is a Tn5 transposase, a Mu transposase, an IS5, or an IS91 transposase.

30. (canceled)

31. (canceled)
